(19) United States
(12) Patent Application Publication
(10) Pub. No.: US 2009/0137687 A1
(43) Pub. Date: May 28, 2009

(54) Compositions and Methods With Enhanced Therapeutic Activity

(75) Inventors: David Chaplin, Oxfordshire (GB); Kevin G. Pinney, Woodway, TX (US); Peter Wardman, Amersham (GB); Vani P. Mocharla, Los Angeles, CA (US); Lisa K. Folkes, Buckinghamshire (GB)

Correspondence Address: LAHIVE & COCKFIELD, LLP;FLOOR 30, SUITE 3000, ONE POST OFFICE SQUARE, BOSTON, MA, 02109, US

(73) Assignees: OXiGENE, Inc., El Granada, CA (US); Baylor University, Waco, TX (US)

(21) Appl. No.: 12/142,495

(22) Filed: Jun. 19, 2008

Related U.S. Application Data
(63) Continuation-in-part of application No. 10/790,662, filed on Mar. 1, 2004.
(60) Provisional application No. 60/936,742, filed on Jun. 21, 2007.
(60) Provisional application No. 60/467,486, filed on May 2, 2003.
(60) Provisional application No. 60/450,565, filed on Feb. 28, 2003.

(51) Int. Cl. A61K 31/122; C07C 49/84; A61K 31/09; C07C 43/23

(52) U.S. Cl. 514/680; 568/326; 568/633; 514/718

(57) ABSTRACT

This invention relates to novel tricyclic quinone and catechol compositions, compositions containing prodrugs of tricyclic quinone and catechol compositions, and methods of use for the treatment of solid tumor cancers and other vascular proliferative disorders. In certain aspects, the compositions of the invention are capable of generating both a vascular targeting effect and tumor cell cytotoxicity (e.g., by oxidative stress) in order to achieve an enhanced anti-tumor response in a patient.

### RELATED APPLICATIONS

This application claims priority to U.S. Provisional Application Ser. No. 60/936,742, entitled “Compositions and Methods with Enhanced Therapeutic Activity”, filed on Jun. 21, 2007. This application also claims priority to U.S. application Ser. No. 10/790,662, entitled “Compositions and Methods with Enhanced Therapeutic Activity”, filed on Mar. 1, 2004, which claims priority to U.S. Provisional Application No. 60/467,486, filed May 2, 2003 and U.S. Provisional Application No. 60/450,565. The entire contents of the aforementioned applications are hereby incorporated herein by reference.

### FIELD OF THE INVENTION

This invention relates to novel tricyclic quinone and catechol compositions, compositions containing prodrugs of tricyclic quinone and catechol compositions, and methods of use for the treatment of solid tumor cancers and other vascular proliferative disorders. In certain aspects, the compositions of the invention are capable of generating both a vascular targeting effect and tumor cell cytotoxicity (e.g., by oxidative stress) in order to achieve an enhanced anti-tumor response in a patient.

### BACKGROUND OF THE INVENTION

Cancer is a leading cause of death in the industrialized world and despite years of research, many types of cancer lack an effective therapeutic treatment. This is especially true for cancers that are characterized by the presence of large, solid tumors, since it is difficult to deliver an effective dose of a chemotherapeutic agent to the interior of a large tumor mass with a significant degree of selectivity. Moreover, due to the genetic instability of tumor cells, a tumor tissue can rapidly acquire resistance to standard therapeutic regimens.

In order to develop into a large solid tumor mass, however, tumor foci must first establish a network of blood vessels in order to obtain the nutrients and oxygen that are required for continued growth. The tumor vascular network has received enormous interest as a therapeutic target for antineoplastic therapy because of its accessibility to blood-borne chemotherapeutics and the relatively small number of blood vessels that are critical for the survival and continued growth of the much larger tumor mass. Disruption in the function of a single tumor blood vessel can result in an avalanche of ischaemic tumor cell death and necrosis of thousands of cancer cells that depend on it for blood supply. In addition, the accessibility of the tumor vasculature to blood-borne anticancer agents and the relatively stable genome of normal, host vascular tissue can alleviate some of the problems such as bioavailability and acquired drug resistance that are associated with conventional, anti-tumor based therapies.

Much of the research in anti-vascular cancer therapy has focused on understanding the process of new blood vessel formation, known as angiogenesis, and identifying anti-angiogenic agents that inhibit the formation of new blood vessels. Angiogenesis is characterized by the proliferation of tumor endothelial cells that form new vasculature to support the growth of a tumor. This growth is stimulated by certain growth factors produced by the tumor itself. One of these growth factors, Vascular Endothelial Growth Factor (“VEGF”), is relatively specific towards endothelial cells, by virtue of the restricted and up-regulated expression of its cognate receptor. Various anti-angiogenic strategies have been developed to inhibit this signaling process at one or more steps in the biochemical pathway in order to prevent the growth and establishment of the tumor vasculature. However, anti-angiogenic therapies act slowly and must be chronically administered over a period of months to years in order to produce a desired effect.

Vascular Targeting Agents (“VTAs”), also known as Vascular Damaging Agents, are a novel class of antineoplastic drugs that exert their effects on solid tumors by selectively occluding, damaging, or destroying the existing tumor vasculature. This disruption of the tumor vasculature occurs rapidly, within minutes to hours following VTA administration, and manifests as a selective reduction in the flow to at least a portion of a tumor region or loss in the number of functional tumor blood vessels in at least a portion of a tumor region, leading eventually to tumor cell death by induction of hypoxia and nutrient depletion. The selectivity of the agent is evidenced by the fact that there are few adverse effects on the function of blood vessels in normal tissues. Thus, the anti-vascular mechanism of VTA action is quite divorced from that of anti-angiogenic agents that do not disrupt existing tumor vasculature but in contrast inhibit molecular signals which induce the formation of tumor neovasculature.

While in vivo studies have confirmed that vascular damaging effects of VTAs on tumor tissue far exceed their effects on normal tissues (Chaplin, et al., Anticancer Research, 1999, 19(1A): 189-196), only in a few cases has a tumor regression or complete tumor response been observed when these agents are used alone as a single agent therapy or monotherapy. The lack of traditional tumor response has been attributed to the rapid recolonization of the necrotic tumor core by a viable rim of tumor cells at the periphery of the tumor capable of receiving oxygen and nutrients from the surrounding normal tissue to resist the effects of blood flow shutdown (Chaplin, et al., Anticancer Research, 1999, 19(1A):189-196). While this viable rim is resistant to VTA therapy, it remains highly susceptible to conventional radiation, chemotherapy and antibody-based therapeutics, and many studies have demonstrated effective tumor regression when VTAs are used in combination with one of these therapies (Li and Rojiani, Int. J. Radiat. Oncol. Biol. Phys., 1998, 42(4): 899-903; Grosios et al., Anticancer Research, 2000, 20(1A): 229-233; Pedley et al., Cancer Research, 2001, 61(12): 4716-4722; WO 02/056692).

Despite the effectiveness when used in combination with VTA therapy, conventional therapies must be administered in repeat daily doses following initial VTA administration in order to achieve prolonged tumor regression. Most conventional therapies are highly cytotoxic, and the patient must cope with prolonged side effects (emesis, hair loss, myelosuppression, etc.) due to chronic administration. VTA therapies lack many of these toxic effects. There is therefore an urgent need in the art for a VTA compound which can be used effectively as a single agent and has the capacity to destroy tumor cells in all regions of the tumor, including the periphery.

### SUMMARY OF THE INVENTION

In a first aspect, the invention provides compositions that selectively reduce blood flow to a tumor region and form a ROS in vivo. The compositions include an anticancer agent having a quinone, quinone prodrug, catechol or catechol prodrug moiety.

In a preferred embodiment, invention provides compounds of formula I:

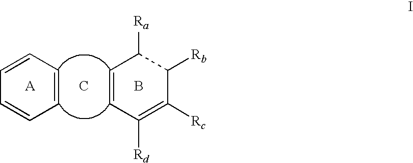

In a more preferred embodiment, the invention provides compounds of formula I-A:

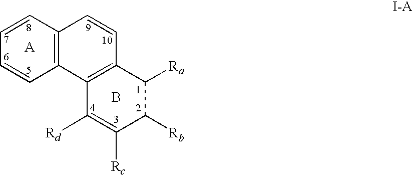

where the dashed line in ring B can be either a single or double bond, when the dashed line is a single bond, both Rₐ and R_{b} are ═O forming a quinone; when the dashed line in ring B is a double bond both Rₐ and R_{b} are as defined below.

In one embodiment, a compound of the invention is a tricyclic catechol which is oxidatively activated in the body to form a quinone which can participate in a redox cycling reaction and form one or more Reactive Oxygen Species (ROS). In another embodiment, a compound of the invention is a tricyclic quinine which can participate in redox cycling and form one or more ROS.

In a second aspect, the present invention provides prodrug compounds of the aforementioned catechols and quinone compositions.

In a third aspect, the invention provides a method of inhibiting the proliferation of tumor cells in a patient bearing a solid tumor comprising administering to the patient an effective amount of a catechol or quinone composition or a prodrug thereof.

In a preferred embodiment, the tricyclic catechol or quinone composition is capable of forming Reactive Oxygen Species (“ROS”) in a locality of the tumor, thereby directly inhibiting the proliferation of tumor cells.

In a fourth aspect, the invention provides a method of reducing blood flow in a patient suffering from a vascular proliferative disorder comprising administering to the patient an effective amount of a tricyclic catechol or tricyclic quinine of the invention or a prodrug thereof. In a preferred embodiment the reduction in blood flow causes the occlusion, destruction, or damage of proliferating vasculature in the patient. In a more preferred embodiment, the effect of reduced blood flow is reversible so that blood flow is restored following cessation of compound administration.

In a fifth aspect, the invention provides a method of generating an enhanced anti-tumor effect in a patient bearing a solid tumor comprising the administration of an effective amount of a tricyclic catechol or tricyclic quinine or the invention or prodrug thereof which is capable of both inhibiting the proliferation of tumor cells and reducing the flow of blood to at least a portion of the tumor.

In a sixth aspect, the invention provides the use of a tricyclic catechol or tricyclic quinone composition of the invention or a prodrug composition, for use as an antimicrotubule agent.

### BRIEF DESCRIPTION OF THE DRAWINGS

FIG. 1 illustrates the oxidative metabolism of combretastatin A-1 (CA1) and formation of quinine and catechol metabolites of CA1. A tricyclic quinone of the invention is depicted as Q2. A tricyclic catechol of the invention is depicted as Q2H2.

FIG. 2. illustrates UV/vis spectrum of CA1 (A), and UV/vis and mass spectra (m/z+1) measured by LC/MS of Q1 (dashed line, B), Q2 (dotted line, C) and Q2H₂ (solid line, D).

FIG. 3. illustrates HPLC chromatograms showing ( ) Q1 (peak 1) and Q2 (3) from oxidation of CA1 (2) by FeCl₃/H₂SO₄;); formation of CA1 (2) from the reduction of Q1 with excess ascorbate (5); ( ); and the product (4) from the reaction of GSH with Q1, assigned to Q1H2-SG (see FIG. 1).

FIG. 4. illustrates LC/MS chromatograms monitoring at m/z=638, showing product prepared chemically by reaction of Q1 and GSH, assigned to Q1H₂—SG (□, A); chromatogram from mouse liver homogenate 18 min post administration of CA1P (50 mg/Kg) to a SCID mouse (□, B); and chromatogram from liver homogenate from a control SCID mouse (□, C).

FIG. 5. illustrates depletion of oxygen in air-saturated solutions of Q2 (50 μM) in phosphate buffer (25 mM, pH 7.4) containing DTPA (100 μM) at 37° C. after the addition of: (A) ascorbate (0.3 mM), (B) glutathione (3 mM), or (C) without addition. Insert: HPLC chromatograms at 268 nm showing Q2 (0.5 mM) (□) was reduced to QH₂ (□) by addition of ascorbate (5 mM).

FIG. 6. Top panel: illustrates approximate relative abundances of Q1 (−) and Q2 (,) after increasing times of reaction of CA1 (100 μM) with HRP (6.7 μg/mL) and H₂O₂ (10 μM), monitoring by HPLC at 295 nm. Lower panel: illustrates depletion of oxygen in mixtures of CA1, HRP and H₂O₂ as described above. (A), CA1 and H₂O₂ alone; (B)-(E), CA1, H₂O₂ and HRP. Ascorbate (1 mM) was added either immediately after adding HRP (B) or 1 min (C), 3 min (D) or 10 min (E) after initiating reaction with HRP.

FIG. 7. illustrates EPR spectra of radical(s) obtained from CA1 or Q2. (A)-(D) with 0.2 M MgCl₂: (A) CA1 (0.4 mM), Tris pH 7.4 (40 mM), 2% v/v DMSO; (B) as (A) with 0.1 mg/mL tyrosinase; (C) as (A) with 0.1 mg/mL HRP; (D) 0.5 mM Q2 in ? pH 7.4, 10% v/v MeCN. (E) Simulated spectrum with a_{N}=0.481 mT, aH=0.153, 0.081 (3) and 0.066 mT, linewidth 0.027 mT, lineshape=86% Lorentzian. (F) Without MgCl₂: CA1 (0.4 mM), Tris (pH 7.4), tyrosinase (0.1 mg/mL), 37° C.

FIG. 8. illustrates EPR spectra obtained from Q2 in the presence of DMPO (0.2 M), EtOH (10% v/v), DTPA (2 mM), phosphate (? M, pH 7.4): (A) GSH (5 mM), no Q2; (B) GSH (5 mM) and Q2 (50 μM) after 15 min; (C) simulation of (B) with species 1=75% (a_{N}=a_{H}=0.149 mT), species 2=15% (a_{N}=0.159 mT, a_{H}=0.229 mT) and species 3=10% (a_{N}=0.157 mT). (D) ascorbate (5 mM) and Q2 (50 μM) after 15 min.

FIG. 9. illustrates (A) Absorption spectra 50 μs after reaction of N₃. with CA1 (,) or CA4 (−), obtained after pulse radiolysis (4.5 Gy) of N20-saturated solutions of the combretastatin (50 μM) with 0.1 M NaN₃, pH 7.4. (B) Absorbance/time traces showing the stability of the radical formed on oxidation of CA1 by N₃. in solutions saturated with N₂O or N₂O:O₂ 80:20 v/v.

FIG. 10. illustrates (A) Absorption spectra 200 μs after reaction of Q2 (30 μM) with either CO₂.⁻ (−) or O₂.⁻ (,), obtained on pulse radiolysis (3 Gy) of solutions containing NaHCO₂ (0.1 M), pH 7.4, saturated with O₂ or N₂O respectively. (B) Absorbance/time traces at 390 under the same conditions.

FIG. 11. illustrates cyclic voltammograms of CA1 (A) CA4 (B) and Q2 (C) at pH 7.4.

### DETAILED DESCRIPTION OF THE INVENTION

The invention is based, at least in part, on the discovery that ortho dihydroxybenzene VTAs (catechol VTAs) such as Combretastatin A-1 (CA1) are susceptible to oxidative metabolism and the formation of free radicals. In addition to their vascular targeting properties, catechol VTAs may be easily oxidized in tumor tissue to form ortho quinones. Ortho-quinones are cytotoxic to the tumor by reacting towards thiols and other biological nucleophiles and forming free radicals thereby causing oxidative stress. In particular, the invention provides novel tricyclic catechol and quinone intermediates (e.g., phenanthrene catechols or phenanthrene ortho-quinones) which are formed by oxidative metabolism of catechol VTAs (e.g., CA1). In certain embodiments, the tricyclic compounds of the invention can be prepared chemically by oxidation of catechol percursors. Reactivity of quinone intermediates of the invention towards glutathione (GSH) was observed in chemical models and confirmed in mice. Evidence for free radical formation and oxygen consumption, as well the interaction with GSH or ascorbate (AscH⁻), demonstrated that the quinone and catechol compounds of the invention undergo redox cycling, which is advantageous property for targeting tumor cell killing.

In a preferred embodiment, invention provides isolated compounds of formula I:

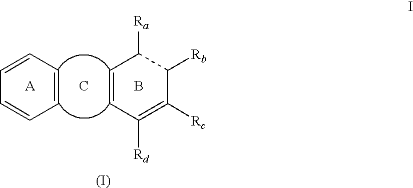

wherein:
(i) Ring A is independently substituted with one to four substituents selected from:
   a C₁, C₂, C₃, C₄ or C₅ branched or straight-chain lower alkoxy, cycloalkoxy, heterocycloalkoxy, aryloxy, or lower alkanoyloxy group; or halogen or trihaloalkyl; or
   a C₁, C₂, C₃, C₄ or C₅ branched or straight chain lower alkyl, allyl, allyloxy, vinyl, or vinyloxy group; or
   OH, or a C₁, C₂, C₃, C₄ or C₅ primary, secondary, or tertiary alcohol;
   an NH₂ or an amino, lower alkylamino, arylamino, aralkylamino, cycloalkylamino, heterocycloamino, aroylamino, aralkanoylamino, amido, lower alkylamido, arylamido, aralkylamido, cycloalkylamido, heterocycloamido, aroylamido, or aralkanoylamido; or
   a lower alkanoyl, thiol, sulfonyl, sulfonamide, nitro, nitrosyl, cyano, carboxy, aryl, or heterocyclo;
(ii) the dashed line of ring B is a single or double bond;
when the dashed line is a double bond, Rₐ and R_{b} are each independently:
   a C₁, C₂, C₃, C₄ or C₅ branched or straight-chain lower alkoxy, cycloalkoxy, heterocycloalkoxy, aryloxy, or lower alkanoyloxy group; or
halogen or trihaloalkyl; or
   a C₁, C₂, C₃, C₄ or C₅ branched or straight chain lower alkyl, allyl, allyloxy, vinyl, or vinyloxy group; or
   OH or a C₁, C₂, C₃, C₄ or C₅ primary, secondary, or tertiary alcohol; or
   NH₂ or an amino, lower alkylamino, arylamino, aralkylamino, cycloalkylamino, heterocycloamino, aroylamino, aralkanoylamino, amido, lower alkylamido, arylamido, aralkylamido, cycloalkylamido, heterocycloamido, aroylamido, or aralkanoylamido; or
   lower alkanoyl, thiol, sulfonyl, sulfonamide, nitro, nitrosyl, cyano, carboxy, aryl, or heterocyclo;
with the proviso that when Rₐ is H, R_{b} is not OH;
when the dashed line is a single bond, Rₐ and R_{b} are each, independently, C═O; and R_{c} and R_{d} of Ring B are each, independently:
hydrogen, or a C₁, C₂, C₃, C₄ or C₅ branched or straight-chain lower alkoxy, cycloalkoxy, heterocycloalkoxy, aryloxy, or lower alkanoyloxy group; or
halogen or trihaloalkyl;
a C₁, C₂, C₃, C₄ or C₅ branched or straight chain lower alkyl, allyl, allyloxy, vinyl, or vinyloxy group; or
OH or C₁, C₂, C₃, C₄ or C₅ primary, secondary, or tertiary alcohol; or
NH₂ or an amino, lower alkylamino, arylamino, aralkylamino, cycloalkylamino, heterocycloamino, aroylamino, aralkanoylamino, amido, lower alkylamido, arylamido, aralkylamido, cycloalkylamido, heterocycloamido, aroylamido, or aralkanoylamido; or lower alkanoyl, thiol, sulfonyl, sulfonamide, nitro, nitrosyl, cyano, carboxy, aryl, or heterocyclo;
(ii) Ring C is an aromatic or non-aromatic, carbocyclic or heterocyclic, 5, 6, or 7 membered ring, optionally substituted with substituents selected from:
a C₁, C₂, C₃, C₄ or C₅ branched or straight-chain lower alkoxy, hydrogen, cycloalkoxy, heterocycloalkoxy, aryloxy, or lower alkanoyloxy group; or
halogen or trihaloalkyl; or
a C₁, C₂, C₃, C₄ or C₅ branched or straight chain lower alkyl, allyl, allyloxy, vinyl, or vinyloxy group; or
OH, or a C₁, C₂, C₃, C₄ or C₅ primary, secondary, or tertiary alcohol; or
NH₂ or an amino, lower alkylamino, arylamino, aralkylamino, cycloalkylamino, heterocycloamino, aroylamino, aralkanoylamino, amido, lower alkylamido, arylamido, aralkylamido, cycloalkylamido, heterocycloamido, aroylamido, or aralkanoylamido; or
lower alkanoyl, thiol, sulfonyl, sulfonamide, nitro, nitrosyl, cyano, carboxy, aryl, or heterocyclo.

In a more preferred embodiment, the invention provides isolated compounds comprising the structure of Formula I-A:

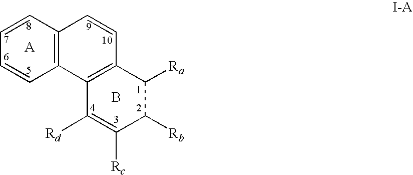

wherein:

(i) Ring A is independently substituted with one to four substituents selected from:
a C₁, C₂, C₃, C₄ or C₅ branched or straight-chain lower alkoxy, cycloalkoxy, heterocycloalkoxy, aryloxy, or lower alkanoyloxy group; or

halogen or trihaloalkyl; or
a C₁, C₂, C₃, C₄ or C₅ branched or straight chain lower alkyl, allyl, allyloxy, vinyl, or vinyloxy group; or
OH, or a C₁, C₂, C₃, C₄ or C₅ primary, secondary, or tertiary alcohol;
an NH₂ or an amino, lower alkylamino, arylamino, aralkylamino, cycloalkylamino, heterocycloamino, aroylamino, aralkanoylamino, amido, lower alkylamido, arylamido, aralkylamido, cycloalkylamido, heterocycloamido, aroylamido, or aralkanoylamido; or
a lower alkanoyl, thiol, sulfonyl, sulfonamide, nitro, nitrosyl, cyano, carboxy, aryl, or heterocyclo;

(ii) the dashed line of ring B is a single or double bond;

when the dashed line is a double bond, Rₐ and R_{b} are each independently:

a C₁, C₂, C₃, C₄ or C₅ branched or straight-chain lower alkoxy, cycloalkoxy, heterocycloalkoxy, aryloxy, or lower alkanoyloxy group; or

halogen or trihaloalkyl; or

a C₁, C₂, C₃, C₄ or C₅ branched or straight chain lower alkyl, allyl, allyloxy, vinyl, or vinyloxy group; or

OH or a C₁, C₂, C₃, C₄ or C₅ primary, secondary, or tertiary alcohol; or

NH₂ or an amino, lower alkylamino, arylamino, aralkylamino, cycloalkylamino, heterocycloamino, aroylamino, aralkanoylamino, amido, lower alkylamido, arylamido, aralkylamido, cycloalkylamido, heterocycloamido, aroylamido, or aralkanoylamido; or lower alkanoyl, thiol, sulfonyl, sulfonamide, nitro, nitrosyl, cyano, carboxy, aryl, or heterocyclo;

with the proviso that when Rₐ is H, R_{b} is not OH;

when the dashed line is a single bond, Rₐ and R_{b} are each, independently, C═O; and

R_{c} and R_{d} of Ring B are each, independently:

hydrogen, or a C₁, C₂, C₃, C₄ or C₅ branched or straight-chain lower alkoxy, cycloalkoxy, heterocycloalkoxy, aryloxy, or lower alkanoyloxy group; or

halogen or trihaloalkyl;

a C₁, C₂, C₃, C₄ or C₅ branched or straight chain lower alkyl, allyl, allyloxy, vinyl, or vinyloxy group; or

OH or C₁, C₂, C₃, C₄ or C₅ primary, secondary, or tertiary alcohol; or

NH₂ or an amino, lower alkylamino, arylamino, aralkylamino, cycloalkylamino, heterocycloamino, aroylamino, aralkanoylamino, amido, lower alkylamido, arylamido, aralkylamido, cycloalkylamido, heterocycloamido, aroylamido, or aralkanoylamido; or

lower alkanoyl, thiol, sulfonyl, sulfonamide, nitro, nitrosyl, cyano, carboxy, aryl, or heterocyclo; and

Ring C is independently substituted with one to two substituents selected from:

a C₁, C₂, C₃, C₄ or C₅ branched or straight-chain lower alkoxy, hydrogen, cycloalkoxy, heterocycloalkoxy, aryloxy, or lower alkanoyloxy group; or

halogen or trihaloalkyl; or

a C₁, C₂, C₃, C₄ or C₅ branched or straight chain lower alkyl, allyl, allyloxy, vinyl, or vinyloxy group; or

OH, or a C₁, C₂, C₃, C₄ or C₅ primary, secondary, or tertiary alcohol;

an NH₂ or an amino, lower alkylamino, arylamino, aralkylamino, cycloalkylamino, heterocycloamino, aroylamino, aralkanoylamino, amido, lower alkylamido, arylamido, aralkylamido, cycloalkylamido, heterocycloamido, aroylamido, or aralkanoylamido; or

a lower alkanoyl, thiol, sulfonyl, sulfonamide, nitro, nitrosyl, cyano, carboxy, aryl, or heterocyclo.

In one embodiment, Ring A of Formula I-A is substituted with one, two, three or four methoxy groups.

In another embodiment, R_{c} and R_{d} of Formula I-A are each, independently, hydrogen or a methoxy group.

In a still another embodiment, when dashed line of ring B of Formula I-A is a single bond;

Rₐ and R_{b} are both ═O;

Ring A is optionally substituted with one to five substituents selected from:

a C₁, C₂, C₃, C₄ or C₅ branched or straight-chain lower alkoxy, cycloalkoxy, heterocycloalkoxy, aryloxy, or lower alkanoyloxy group; or

a C₁, C₂, C₃, C₄ or C₅ branched or straight chain lower alkyl, allyl, allyloxy, vinyl, or vinyloxy group;

R_{c} is selected from:

a C₁, C₂, C₃, C₄ or C₅ branched or straight-chain lower alkoxy, cycloalkoxy, heterocycloalkoxy, aryloxy, or lower alkanoyloxy group; or

a C₁, C₂, C₃, C₄ or C₅ branched or straight chain lower alkyl, allyl, allyloxy, vinyl, or vinyloxy group; and

R_{d} is hydrogen.

In a yet another embodiment, when the dashed line of ring B of Formula I-A is a double bond;

Ring A is optionally substituted with one to five substituents selected from:

a C₁, C₂, C₃, C₄ or C₅ branched or straight-chain lower alkoxy, cycloalkoxy, heterocycloalkoxy, aryloxy, or lower alkanoyloxy group; or
a C₁, C₂, C₃, C₄ or C₅ branched or straight chain lower alkyl, allyl, allyloxy, vinyl, or vinyloxy group;

Rₐ and R_{b} are both OH;

R_{c} is selected from:

a C₁, C₂, C₃, C₄ or C₅ branched or straight-chain lower alkoxy, cycloalkoxy, heterocycloalkoxy, aryloxy, or lower alkanoyloxy group; or

a C₁, C₂, C₃, C₄ or C₅ branched or straight chain lower alkyl, allyl, allyloxy, vinyl, or vinyloxy group; and

R_{d} is hydrogen.

In another embodiment, the compound of formula I-A is substituted with methoxy groups in the 3, 5, 6, and 7 positions.

In another embodiment, the invention provides isolated compounds comprising the structure of Formula I-B:

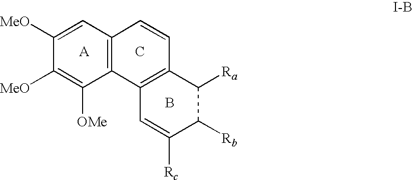

wherein:

the dashed line of ring B is a single or double bond;

when the dashed line is a double bond, Rₐ and R_{b} are each independently:

a C₁, C₂, C₃, C₄ or C₅ branched or straight-chain lower alkoxy, cycloalkoxy, heterocycloalkoxy, aryloxy, or lower alkanoyloxy group; or

a C₁, C₂, C₃, C₄ or C₅ branched or straight chain lower alkyl, allyl, allyloxy, vinyl, or vinyloxy group; or

OH or a C₁, C₂, C₃, C₄ or C₅ primary, secondary, or tertiary alcohol;

with the proviso that when Rₐ is H, R_{b} is not OH;

when the dashed line is a single bond, Rₐ and R_{b} are each, independently, C═O; and

R_{c} of Ring B is:

hydrogen, or a C₁, C₂, C₃, C₄ or C₅ branched or straight-chain lower alkoxy, cycloalkoxy, heterocycloalkoxy, aryloxy, or lower alkanoyloxy group; or

a C₁, C₂, C₃, C₄ or C₅ branched or straight chain lower alkyl, allyl, allyloxy, vinyl, or vinyloxy group; or

OH or C₁, C₂, C₃, C₄ or C₅ primary, secondary, or tertiary alcohol;

and

Ring C is independently substituted with one to two substituents selected from:

a C₁, C₂, C₃, C₄ or C₅ branched or straight-chain lower alkoxy, hydrogen, cycloalkoxy, heterocycloalkoxy, aryloxy, or lower alkanoyloxy group; or

halogen or trihaloalkyl; or

a C₁, C₂, C₃, C₄ or C₅ branched or straight chain lower alkyl, allyl, allyloxy, vinyl, or vinyloxy group; or

OH, or a C₁, C₂, C₃, C₄ or C₅ primary, secondary, or tertiary alcohol;

an NH₂ or an amino, lower alkylamino, arylamino, aralkylamino, cycloalkylamino, heterocycloamino, aroylamino, aralkanoylamino, amido, lower alkylamido, arylamido, aralkylamido, cycloalkylamido, heterocycloamido, aroylamido, or aralkanoylamido; or

a lower alkanoyl, thiol, sulfonyl, sulfonamide, nitro, nitrosyl, cyano, carboxy, aryl, or heterocyclo.

In still another embodiment, the invention provides isolated compounds comprising the structure of Formula I-B wherein the compound of formula I-B is selected from the group consisting of 3,5,6,7-tetramethoxyphenanthrene-1,2-dione (1) and 3,5,6,7-tetramethoxyphenanthrene-1,2-diol (2).

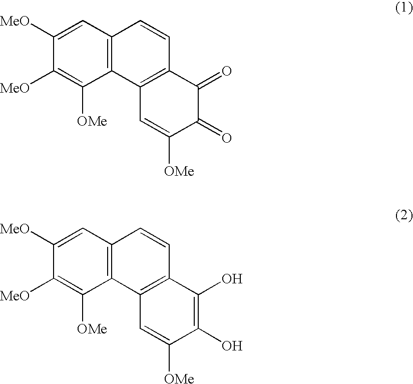

In another embodiment, the invention provides a method for selectively reducing blood flow to a tumor region and forming a ROS in a patient suffering from cancer, comprising administering a compound of Formulas I, I-A or I-B.

In yet another embodiment, the invention provides a method of inhibiting the proliferation of tumor cells in a patient suffering from cancer, comprising administering to the patient an effective amount of a compound of Formulas I, I-A or I-B.

In still another embodiment, the invention provides a method of reducing blood flow in a patient suffering from a vascular proliferative disorder, comprising administering to the patient an effective amount of a compound of Formulas I, I-A or I-B.

In another embodiment, the invention provides a pharmaceutical composition comprising the compound of any one of Formulas I, I-A or I-B in a pharmaceutically acceptable carrier.

### DEFINITIONS

As used herein, the following terms in quotations shall have the indicated meanings, whether in plural or singular form.

“Alkyl” when used alone or in combination with other groups, includes lower alkyl containing from 1 to 8 carbon atoms and may be straight chained or branched. An alkyl group includes optionally substituted straight, branched or cyclic saturated hydrocarbon groups. When substituted, alkyl groups may be substituted with up to four substituent groups, R as defined, at any available point of attachment. When the alkyl group is said to be substituted with an alkyl group, this is used interchangeably with “branched alkyl group”. Exemplary unsubstituted such groups include methyl, ethyl, propyl, isopropyl, n-butyl, t-butyl, isobutyl, pentyl, hexyl, isohexyl, heptyl, 4,4-dimethylpentyl, octyl, 2,2,4-trimethylpentyl, nonyl, decyl, undecyl, dodecyl, and the like. Exemplary substituents may include, but are not limited to one or more of the following groups: halo (such as F, Cl, Br, I), haloalkyl (such as CCl₃ or CF₃), alkoxy, alkylthio, hydroxy, carboxy (—COOH), alkyloxycarbonyl (—C(O)R), alkylcarbonyloxy (—OCOR), amino (—NH₂), carbamoyl (—NHCOOR— or —OCONHR—), urea (—NHCONHR—) or thiol (—SH). Alkyl groups as defined may also comprise one or more carbon-carbon double bonds or one or more carbon-carbon triple bonds.

Preferred alkyl groups contain 1-8 carbon atoms; more preferred alkyl groups contain 1-6 carbon atoms. Alkylene as used herein includes a bridging alkyl group of the formula CₙH₂ₙ. Examples include CH₂, —CH₂CH₂—, —CH₂ CH₂CH₂— and the like.

As used herein the term “cycloalkyl” is a species of alkyl containing from 3 to 15 carbon atoms, without alternating or resonating double bonds between carbon atoms. It may contain from 1 to 4 rings. Exemplary unsubstituted such groups include cyclopropyl, cyclobutyl, cyclopentyl, cyclohexyl, adamantyl, etc. Exemplary substituents include one or more of the following groups: halogen, alkyl, alkoxy, alkyl hydroxy, amino, nitro, cyano, thiol and/or alkylthio.

“Aryl” refers to groups with aromaticity, including 5- and 6-membered single-ring aromatic groups that may include from zero to four heteroatoms, as well as multicyclic systems with at least one aromatic ring. Examples of aryl groups include benzene, phenyl, heterocyclic groups (pyrrole, furan, thiophene, thiazole, isothiazole, imidazole, indole, morpholine, triazole, thiene, tetrazole, pyrazole, oxadiozole, oxazole, isooxazole, piperidine, pyridine, pyrazine, pyridazine, and pyrimidine, and the like), bicyclic heterocyclic groups (benzothiazole, benzothiene, quinoline, isoquinoline, benzaimidazole, benzopyrane, indolizine, benzofuran, chromine, courmain, cinnoline, quinoxaline, indazole, pyrrolopyridine, furopyridine, naphthalene, dihydroisoindoline, dihydroquinazoline, benzisothiazole, benzopyrazole, dihydrobenzofurane, dihydrobenzothiene, dihydronaphthalene, dihydrobenzopyrane, phthalazine, purine, and the like), and polycyclic groups (anthracene, phenanthrene, chrysene, and the like). The aromatic ring can be substituted at one or more ring positions with such substituents as described above, as for example, halogen, hydroxyl, alkoxy, etc. The preferred aryl group of the present invention is a benzene ring.

“Cancer”, “Neoplastic Disease”, and “Tumor” shall be used interchangeably and shall refer to the abnormal presence of cells which exhibit relatively autonomous growth, so that they exhibit an aberrant growth phenotype characterized by a significant loss of cell proliferation control. Cancerous cells can be benign or malignant. In various embodiments, the cancer affects cells of the bladder, blood, brain, breast, colon, digestive tract, lung, ovaries, pancreas, prostate gland, thyroid, or skin.
a) solid carcinomas, including cancers of the lung (such as small cell lung cancer, non-small cell lung cancer, and lung adenocarcinoma), colon (including colorectal cancer), ovaries, prostrate, testes, cervix, genitourinary tract, bladder (including accelerated and metastatic bladder cancer), liver, larynx, esophagus, stomach, breast, kidney, gall bladder, thyroid, pancreas (including exocrine pancreatic carcinoma), and skin (including squamous cell carcinoma);
b) hematopoietic tumors of lymphoid lineage, including leukemia, acute lymphocytic leukemia, acute lymphoblastic leukemia, B-cell lymphoma, T-cell lymphoma, Hodgkins lymphoma, non-Hodgkins lymphoma, hairy cell lymphoma and Burkett's lymphoma;
c) hematopoietic tumors of myeloid lineage, including acute and chronic myelogenous leukemias, myelodysplastic syndrome, and promyelocytic leukemia;
d) tumors of mesenchymal origin, including fibrosarcoma, osteosarcoma and rhabdomyosarcoma;
e) tumors of the central and peripheral nervous system, including astrocytoma, neuroblastoma, glioma and schwannomas; and
f) other tumors, including melanoma, seminoma, teratocarcinoma, osteosarcoma, xenoderoma pigmentosum, keratoactanthoma, thyroid follicular cancer, medullary thyroid cancer, anaplastic thyroid cancer, teratocarcinoma, and Kaposi's sarcoma.

“Antiproliferative” refers to the ability of the compounds of the present invention to directly inhibit tumor cells from multiplying. In general, the antiproliferative activity of the compositions of the invention fall into two classes, anti-proliferative cytotoxic and anti-proliferative cytostatic. Cytotoxic agents prevent tumor cells from multiplying by: (1) directly interfering with the ability of tumor cells to replicate DNA or undergo mitotic cell division and (2) inducing cell death and/or apoptosis in the cancer cells. Anti-proliferative cytostatic or quiescent agents act via modulating, interfering or inhibiting the processes of cellular signal transduction which regulate cell proliferation in order to slow the rate of cell division or tumor growth so that the cells become non-proliferative or so that their behavior approximates that of non-proliferative cells.

“Catechol” is any group of optionally substituted compounds with aryl functionality and containing at least two OH groups the ortho position or para position on the Aryl ring, wherein a conjugated system is formed with at least one C═C bond. The preferred catechol of the present invention is an ortho-benzocatechol.

“Effective Amount” shall be an amount of drug which generates a significant anti-tumor effect including but not limited to, inhibition of tumor growth, tumor growth delay, tumor regression, tumor shrinkage, increased time to regrowth of tumor on cessation of treatment, and slowing of disease progression. It is expected that when a method of treatment of the present invention is administered to a patient in need of treatment for cancer, said method of treatment will produce an effect, as measured by, for example, one or more of: the extent of the anti-tumor effect, the response rate, the time to disease progression, and the survival rate.

“Halogen” or “Halo” refers to chlorine, bromine, fluorine or iodine.

“Lower alkoxy” refers to —O-alkyl groups, wherein alkyl is as defined hereinabove. The alkoxy group is bonded to the main chain, aryl or heteroaryl group through the oxygen bridge. The alkoxy group may be straight chained or branched; although the straight-chain is preferred. Examples include methoxy, ethyloxy, propoxy, butyloxy, t-butyloxy, i-propoxy, and the like. Preferred alkoxy groups contain 1-4 carbon atoms, especially preferred alkoxy groups contain 1-3 carbon atoms. The most preferred alkoxy group is methoxy.

“Lower alkylamino” refers to a group wherein one alkyl group is bonded to an amino nitrogen, i.e., NH(alkyl). The NH is the bridge connecting the alkyl group to the aryl or heteroaryl. Examples include NHMe, NHEt, NHPr, and the like.

“Proliferating Vasculature” refers to either a tumor vasculature or non-malignant proliferating vasculature, otherwise known as neovasculature or immature vasculature, which supply blood to tumors or normal tissues for the provision of oxygen and nutrients. Proliferating vasculature exhibits structural and functional features that distinguishes it from normal vasculature, including irregular vessel diameter, leakiness, vessel tortuosity, thin vessel wall thickness, heterogeneous blood flow distribution, high interstitial fluid pressure, procoagulant status, or small numbers of supportive cells.

“Quinone” is any group of optionally substituted aromatic polyketone compounds derived from a compound with an Aryl moeity. At least two C═O groups are in the ortho or para position on the Aryl ring, and form a conjugated system with at least one C═C bond. The preferred quinone of the present invention is an ortho-benzoquinone. quinones synthesized in a number of ways by oxidation of a phenolic precursor such as ortho-catechol. The oxidant reagents used in the reaction can include Jones reagent (Chromate salts), Fremy's salt ((KSO₃)₂NO), and the like. The preferred oxidant is o-iodoxybenzoic acid.

“Salt” is a pharmaceutically acceptable salt, i.e., substantially non-toxic and with the desired pharmacokinetic properties, palatability, and solubility, and can include acid addition salts including amino acids, hydrochlorides, hydrobromides, phosphates, sulphates, hydrogen sulphates, alkylsulphonates, arylsulphonates, acetates, ascorbates, benzoates, citrates, glycolates, maleates, nitrates, fumarates, stearates, salicylates, succinates, oxalates, lactates, and tartrates; alkali metal cations such as Na, K, Li, alkali earth metal salts such as Mg or Ca; or organic bases dicyclohexylamine, trbutylamine, pyridine, triethylamine, and as others disclosed in PCT International Application Nos. WO02/22626 or WO00/48606. The salts of the present invention can be synthesized by conventional chemical methods. Generally, the salts are prepared by reacting the free base or acid with stoichiometic amounts or with an excess of the desired salt-forming inorganic or organic acid or base, in a suitable solvent or solvent combination.

“Tubulin Binding Agent” shall refer to a ligand of tubulin or a compound capable of binding α or β-tubulin monomers, αβ-tubulin heterodimers, or polymerized microtubules and interfering with the polymerization or depolymerization of microtubules. The exact nature of tubulin binding site interactions remain largely unknown, and they definitely vary between each class of Tubulin Binding Agent. Photoaffinity labeling and other binding site elucidation techniques have identified three key binding sites: 1) the Colchicine site (Floyd et al, Biochemistry, 1989; Staretz et al, J. Org. Chem., 1993; Williams et al, J. Biol. Chem., 1985; Wolff et al, Proc. Natl. Acad. Sci. U.S.A., 1991), 2) the Vinca Alkaloid site (Safa et al, Biochemistry, 1987), and 3) a site on the polymerized microtubule to which taxol binds (Rao et al, J. Natl. Cancer Inst., 1992; Lin et al, Biochemistry, 1989; Sawada et al, Bioconjugate Chem, 1993; Sawada et al, Biochem. Biophys. Res. Commun., 1991; Sawada et al, Biochem. Pharmacol., 1993). Tubulin binding agents contemplated by the present invention contain at least one aryl moiety where a catechol or quinone structure can be introduced in order to generate a “Dual activity” agent. Particularly preferred tubulin binding agents include:
a) Combretastatins or other stilbene analogs (Pettit et al, Can. J. Chem., 1982; Pettit et al, J. Org. Chem., 1985; Pettit et al, J. Nat. Prod., 1987; Lin et al, Biochemistry, 1989; Singh et al, J. Org. Chem., 1989; Cushman et al, J. Med. Chem., 1991; Getahun et al, J. Med. Chem., 1992; Andres et al, Bioorg. Med. Chem. Lett., 1993; Mannila, Liebigs. Ann. Chem., 1993; Shirai et al, Bioorg. Med. Chem. Lett., 1994; Medarde et al., Bioorg. Med. Chem. Lett., 1995; Pettit et al, J. Med. Chem., 1995; Wood et al, Br. J. Cancer., 1995; Bedford et al, Bioorg. Med. Chem. Lett., 1996; Dorr et al, Invest. New Drugs, 1996; Jonnalagadda et al., Bioorg. Med. Chem. Lett., 1996; Shirai et al, Heterocycles, 1997; Aleksandrzak K, Anticancer Drugs, 1998; Chen et al, Biochem. Pharmacol., 1998; Ducki et al, Bioorg. Med. Chem. Lett., 1998; Hatanaka et al, Bioorg. Med. Chem. Lett., 1998; Medarde, Eur. J. Med. Chem., 1998; Medina et al, Bioorg. Med. Chem. Lett., 1998; Ohsumi et al, Bioorg. Med. Chem. Lett., 1998; Ohsumi et al., J. Med. Chem., 1998; Pettit G R et al., J. Med. Chem., 1998; Shirai et al, Bioorg. Med. Chem. Lett., 1998; Banwell et al, Aust. J. Chem., 1999; Medarde et al, Bioorg. Med. Chem. Lett., 1999; Shan et al, PNAS, 1999; Combeau et al, Mol. Pharmacol, 2000; Pettit et al, J. Med Chem, 2000; Pettit et al, Anticancer Drug Design, 2000; Pinney et al, Bioorg. Med. Chem. Lett., 2000; Flynn et al., Bioorg. Med. Chem. Lett., 2001; Gwaltney et al, Bioorg. Med. Chem. Lett., 2001; Lawrence et al, 2001; Nguyen-Hai et al, Bioorg. Med. Chem. Lett., 2001; Xia et al, J. Med. Chem., 2001; Tahir et al., Cancer Res., 2001; Wu-Wong et al., Cancer Res., 2001; Janik et al, Bioorg. Med. Chem. Lett., 2002; Kim et al., Bioorg Med Chem. Lett., 2002; Li et al, Bioorg. Med. Chem. Lett., 2002; Nam et al, Bioorg. Med. Chem. Lett., 2002; Wang et al, J. Med. Chem. 2002; Hsieh et al, Bioorg. Med. Chem. Lett., 2003; Hadimani et al., Bioorg. Med. Chem. Lett., 2003; Mu et al, J. Med. Chem., 2003; Nam, Curr. Med. Chem., 2003; Pettit et al, J. Med. Chem., 2003; WO 02/50007, WO 02/22626, WO 02/14329, WO 01/81355, WO 01/12579, WO 01/09103, WO 01/81288, WO 01/84929, WO 00/48591, WO 00/48590, WO 00/73264, WO 00/06556, WO 00/35865, WO 00/48590, WO 99/51246, WO 99/34788, WO 99/35150, WO 99/48495, WO 92/16486, U.S. Pat. Nos. 6,433,012, 6,201,001, 6,150,407, 6,169,104, 5,731,353, 5,674,906, 5,569,786, 5,561,122, 5,430,062, 5,409,953, 5,525,632, 4,996,237 and 4,940,726 and U.S. patent application Ser. No. 10/281,528);
b) 2,3-substituted Benzo[b]thiophenes (Pinney et al, Bioorg. Med. Chem. Lett., 1999; Chen et al, J. Org. Chem., 2000; U.S. Pat. Nos. 5,886,025; 6,162,930, and 6,350,777; WO 98/39323);
c) 2,3-disubstituted Benzo[b]furans (WO 98/39323, WO 02/060872);
d) Disubstituted Indoles (Gastpar R, J. Med. Chem., 1998; Bacher et al, Cancer Res., 2001; Flynn et al, Bioorg. Med. Chem. Lett, 2001; WO 99/51224, WO 01/19794, WO 01/92224, WO 01/22954; WO 02/060872, WO 02/12228, WO 02/22576, and U.S. Pat. No. 6,232,327);
e) 2-Aroylindoles (Mahboobi et al, J. Med. Chem., 2001; Gastpar et al., J. Med. Chem., 1998; WO 01/82909)
f) 2,3-disubstituted Dihydronaphthalenes (WO 01/68654, WO 02/060872);
g) Benzamidazoles (WO 00/41669);
h) Chalcones (Lawrence et al, Anti-Cancer Drug Des, 2000; WO 02/47604)
i) Colchicine, Allocolchicine, Thiocolcichine, Halichondrin B, and Colchicine derivatives (WO 99/02166, WO 00/40529, WO 02/04434, WO 02/08213, U.S. Pat. Nos. 5,423,753. 6,423,753) in particular the N-acetyl colchinol prodrug, ZD-6126;
j) Curacin A and its derivatives (Gerwick et al, J. Org. Chem., 1994, Blokhin et al, Mol. Pharmacol., 1995; Verdier-Pinard, Arch. Biochem. Biophys., 1999; WO 02/06267);
k) Dolastatins such as Dolastatin-10, Dolastatin-15, and their analogs (Pettit et al, J. Am. Chem. Soc., 1987; Bai et al, Mol. Pharmacol, 1995; Pettit et al, Anti-Cancer Drug Des., 1998; Poncet, Curr. Pharm. Design, 1999; WO 99/35164; WO 01/40268; U.S. Pat. No. 5,985,837);
m) Epothilones such as Epothilones A, B, C, D and Desoxyepothilones A and B (WO 99/02514, U.S. Pat. No. 6,262,094, Nicolau et al., Nature, 1997);
n) Inadones (Leoni et al., J. Natl. Cancer Inst., 2000; U.S. Pat. No. 6,162,810);
o) Lavendustin A and its derivatives (Mu F et al, J. Med. Chem., 2003);
p) 2-Methoxyestradiol and its derivatives (Fotsis et al, Nature, 1994; Schumacher et al, Clin. Cancer Res., 1999; Cushman et al, J. Med. Chem., 1997; Verdier-Pinard et al, Mol. Pharmacol, 2000; Wang et al, J. Med. Chem., 2000; WO 95/04535, WO 01/30803, WO 00/26229, WO 02/42319 and U.S. Pat. Nos. 6,528,676, 6,271,220, 5,892,069, 5,661,143, and 5,504,074);
q) Monotetrahydrofurans (“COBRAs”; Uckun, Bioorg. Med. Chem. Lett., 2000; U.S. Pat. No. 6,329,420);
r) Phenylhistin and its derivatives (Kanoh et al, J. Antibiot., 1999; Kano et al, Bioorg. Med. Chem., 1999; U.S. Pat. No. 6,358,957);
s) Podophyllotoxins such as Epidophyllotoxin (Hammonds et al, J. Med. Microbiol, 1996; Coretese et al, J. Biol. Chem., 1977);
t) Rhizoxins (Nakada et al, Tetrahedron Lett., 1993; Boger et al, J. Org. Chem., 1992; Rao, et al, Tetrahedron Lett., 1992; Kobayashi et al, Pure Appl. Chem., 1992; Kobayashi et al, Indian J. Chem., 1993; Rao et al, Tetrahedron Lett., 1993);
u) 2-strylquinazolin-4(3H)-ones (“SQOs”, Jiang et al, J. Med. Chem., 1990);
v) Spongistatin and Synthetic spiroketal pyrans (“SPIKETs”; Pettit et al, J. Org. Chem., 1993; Uckun et al, Bioorgn. Med. Chem. Lett., 2000; U.S. Pat. No. 6,335,364, WO 00/00514);
w) Taxanes such as Paclitaxel (Taxol®), Docetaxel (Taxotere®), and Paclitaxel derivatives (U.S. Pat. No. 5,646,176, WIPO Publication No. WO 94/14787, Kingston, J. Nat. Prod., 1990; Schiff et al, Nature, 1979; Swindell et al, J. Cell Biol., 1981);
x) Vinca Alkaloids such as Vinblastine, Vincristine, Vindesine, Vinflunine, Vinorelbine (Navelbine®) (Owellen et al, Cancer Res., 1976; Lavielle et al, J. Med. Chem., 1991; Holwell et al, Br. J. Cancer., 2001); or
y) Welwistatin (Zhang et al, Molecular Pharmacology, 1996).

Many tubulin binding agents have been known to disrupt tumor vasculature but differ in that they also manifest substantial normal tissue toxicity at their maximum tolerated dose. In contrast, genuine VTAs retain their selective tumor vascular shutdown activity at a fraction of their maximum tolerated dose, with minimal effects on normal tumor vasculature. Although tubulin binding agents in general can mediate effects on tumor blood flow, doses that are effective are often also toxic to other normal tissues and not particularly toxic to tumors (Br. J. Cancer 74(Suppl. 27):586-88, 1996). For example, the vascular effects that are observed with colchicines and vinca alkaloids are only evident at doses approximating or surpassing the maximum tolerable dose to the patient (Baguley et al., Eur. J. Cancer., 27(4): 482-487; Hill et al., Eur. J. Cancer, 29A(9): 1320-1324.)

“Tumor microvessel” refers to the endothelium, artery or blood vessel, also known as tumor neovasculature, feeding any type of tumor, whether it be malignant, benign, actively growing, or in remission.

### Compositions:

All stereoisomers of the compounds of the instant invention are contemplated, either in admixture or in pure or substantially pure form. The definition of the compounds according to the invention embraces all possible stereoisomers and their mixtures. It particularly embraces the racemic forms and the isolated optical isomers having the specified activity. The racemic forms can be resolved by physical methods, such as, for example, fractional crystallization, separation or crystallization of diastereomeric derivatives or separation by chiral column chromatography. The individual optical isomers can be obtained from the racemates by conventional methods, such as, for example, salt formation with an optically active acid followed by crystallization.

It should be noted that any heteroatom with unsatisfied valences is assumed to have the hydrogen atom to satisfy the valences.

When a group is referred to as being “Optionally substituted”, it may be substituted with one to five, preferably one to three, substituents such as halogen, alkyl, hydroxyl, lower alkoxy, Amino, Lower alkylamino, cycloalkoxy, heterocycloalkoxy, oxo, lower alkanoyl, aryloxy, lower alkanoyloxy, arylamino, aralkylamino, cycloalkylamino, heterocycloamino, aroylamino, aralkanoylamino, thiol, sulfonyl, sulfonamide, nitro, nitrosyl, cyano, carboxy, carbamyl, aryl, heterocyclo, and the like.

### a) Quinones

The quinones of the present invention were found to participate in a Redox Cycling Reaction and stimulate oxidative stress in tumor cells by the concomitant production of ROS that are directly toxic to tumor cells. In addition, the quinone and semiquinone molecules generated by the oxidation of the catechol may themselves cause tumor cell death by direct cytotoxic mechanisms including membrane damage, lipid peroxidation, and depolymerization of macromolecules. These highly reactive species of catechol can elicit their damage to tumor cells by binding to proteins, lipids, or nucleic acids.

A Redox Cycling Reaction or Oxidation-Reduction reaction is in equilibrium between reduction (increase in electrons) or oxidation (loss of electrons) as illustrated with the following reaction in which ortho-benzoquinone, formed by dephosphorylation of a prodrug, is reductively activated to form its corresponding ortho-catechol which in turn can be oxidized to regenerate the ortho-quinone.

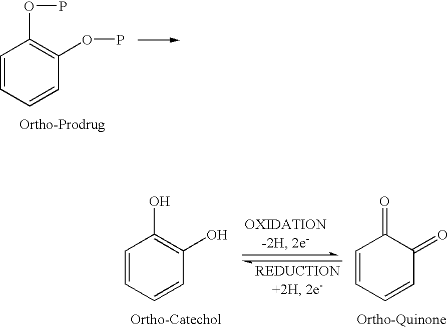

A reduction is facilitated by the oxidation of a reducing agent (electron donor) while oxidation is facilitated by the reduction of an oxidizing agent (electron acceptor).

The quinones of the present invention can be reduced or reductively activated by the presence of a reducing agent such as NADH, NADPH, Ascorbate, Glutathione or reducing enzymes such as the flavoenzyme DT-diaphorase which is highly expressed in many tumor cells.

Oxidative stress induced by the quinones of the present invention is due to the quinone itself or by the formation of Reactive Oxygen Species (ROS) which include Semiquinone radical anion (Q.⁻),

catechol+Reducing Agent→Q.⁻+H⁺+e⁻ (1)

Superoxide radicals (O₂.⁻),

Q.⁻+O₂→Q+O₂.⁻ (2)

Hydrogen peroxide (H₂O₂),

2O₂.⁻+2H+ [symbol] H₂O₂ (3)

or hydroxyl radicals (OH.⁻), if trace heavy metals are present to catalyze their formation from Hydrogen peroxide.

H₂O₂+Reduced Iron/Copper [symbol] .⁻OH+Oxidized Iron/Copper (4)

ROS are directly cytotoxic to tumor cells because they react directly to form adducts with cell components including protein, lipid, and DNA. Alternatively, they can initiate the formation of lipid hydroperoxides which in turn act as mutagens by covalently modifying DNA. Hydroxyl anion radicals, for example, are some of the most powerful oxidants in biological systems and can mediate many destructive mechanisms on tumor cells, including membrane damage, lipid peroxidation, and depolymerization of macromolecules.

### b) Catechols

Catechols of the present invention can be used to generate one or both of the following toxic effects. In the first toxic effect, the catechol compound is able to selectively target endothelial cells of tumor vasculature or other proliferating vasculature and reduce the flow of blood within the proliferating vasculature. The reduction in blood flow can result in damage or regression of the proliferating vasculature and/or inhibition of further vascular proliferation. When administered to an patient bearing a solid tumor, this first toxic effect can result in tumor hypoxia and nutrient deprivation. In the second toxic effect, the catechol is used as a cytotoxic agent which forms its corresponding quinone in vivo and is able to kill tumor cells directly by inducing oxidative stress. In a particularly preferred embodiment, the catechol is a “dual activity” agent capable of eleciting both the first and second toxic effect.

Catechols of the present invention can be activated to form corresponding quinones by the presence of an “oxidizing agent or equivalent”, such as Oxygen or enzymes such as myeloperoxidases or tyrosinases, to form a catechol radical (C.⁻). Formation of the catechol radical establishes a redox cycle in which the production of ROS is amplified multiple times. This is because two catechol radicals can generate an ortho quinone and regenerate the ortho-catechol which can react again to supply additional reactive catechol radicals. Reduction of the quinone by a reducing agent such as NADPH or the enzyme DT-Diaphorase (NADPH quinone-acceptor oxidoreductase), regenerates the original catechol and establishes a redox cycle, which amplifies the formation of ROS.

Catechols thought to be involved in the generation of ROS through redox cycling include:
1) Diols of Polycyclic Aromatic Hydrocarbons (PAH) such as Naphthalene diols, Benz[alpha]anthracene diols, Chrysene diols, Phenanthrene diols, Benz[a]pyrene diols (Sridhar, Tetrahedron, 2001; Flowers-Geary, Chem Biol Interact, 1996), including Menadione.
2) Catechol estrogens or antiestrogens such as 3,4 Dihydroxytamoxifen, Toremifine, Droloxifine, (Bolton, Toxicology, 2002; Chem Res. Toxicol, 2000).
3) Topoisomerase II inhibitors such as Etoposide catechol (Pang, J. of Mass Spec, 2001).

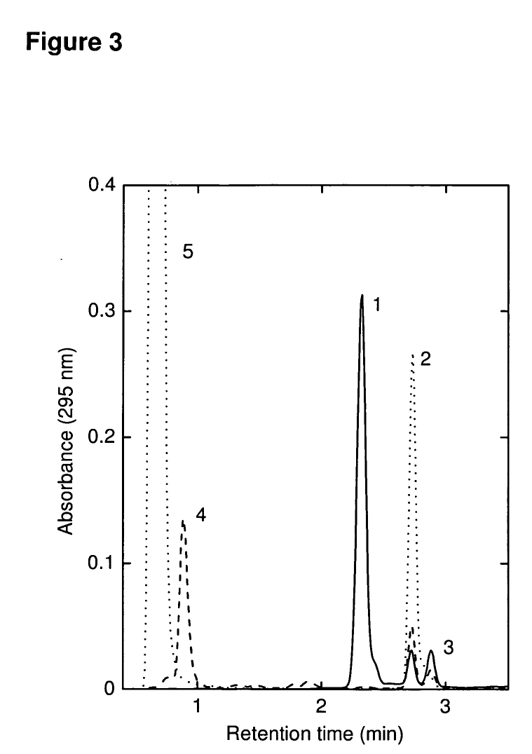

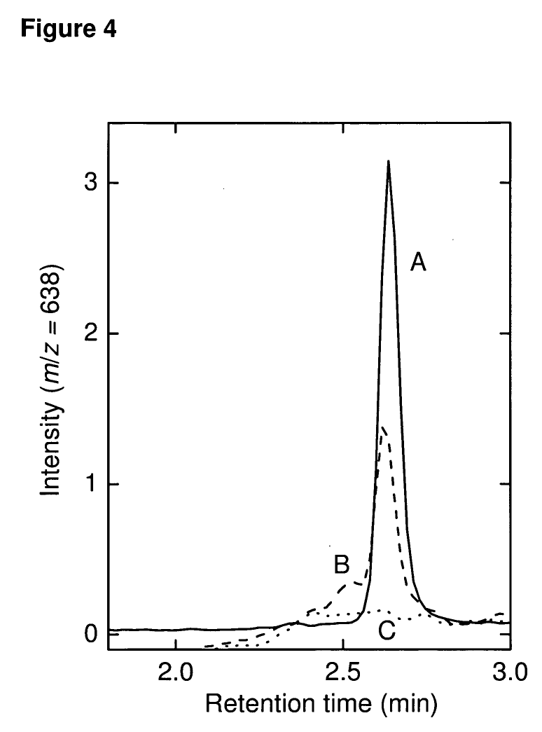

Anticancer agents for use in the present invention contain an aryl functionality and include the following compounds which are classified based on the mechanism of action:
1. Alkylating agents: compounds that donate an alkyl group to nucleotides. Alkylated DNA is unable to replicate itself and cell proliferation is stopped. Exemplary alkylating agents include Melphalan and Chlorambucil. The structure of Melphalan and its corresponding o-quinone are depicted in FIG. 3.
2. Antiangiogeneic agents: compounds that inhibit the formation of tumor vasculature. The structure of an exemplary Alkylating agent, and its corresponding o-quinone are depicted in FIG. 3.
3. Antitumor Antibiotics: compounds having antimicrobial and cytotoxic activity. Such compounds also may interfere with DNA by chemically inhibiting enzymes and mitosis or altering cellular membranes. Exemplary antitumor antibiotics include Dactinomycin, Daunorubicin, and Doxorubicin. The structure of Doxorubucin, and its corresponding o-quinone, are depicted in FIG. 3.
4. Topoisomerase Inhibitors: agents which interfere with topoisomerase activity thereby inhibiting DNA replication. Such agents include CPT-11 and Topotecan. The structure of Topotecan and its corresponding o-quinone is depicted in FIG. 3.
5. Hormonal Therapy: includes, but is not limited to anti-estrogens. An exemplary antiestrogen is Tamoxifen.
6. Mitotic inhibitors: compounds that inhibit mitosis or inhibit enzymes that prevent protein synthesis needed for reproduction of the cell. Preferred mitotic inhibitors are tubulin binding agents. The structure of representative exemplary tubulin binding agents, and their corresponding o-quinones, are depicted in FIG. 4.

### c) Prodrugs

i) Catechol Prodrugs. Prodrugs of the present invention are precursor forms of catechols that are metabolically converted in vivo to produce corresponding catechols. In an important specific sense, to which however the invention is in its broadest aspects not limited, the prodrug in the foregoing methods, compositions and procedures may be a Phosphate within the class of compounds having the general formula

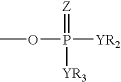

wherein

Y is O, NH, S, O⁻, NH⁻ or S⁻;

Z is O or S; and

each of R² and R³ is an alkyl group, H, a monovalent or divalent metal cationic salt, or an ammonium cationic salt, and R² and R³ may be the same or different.

Currently preferred prodrugs for the practice of the invention are those having the following formulae:

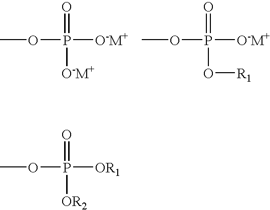

Other prodrugs contemplated for use in the present invention include Sulphates of the following general formula

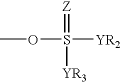

wherein

Y is O, NH, S, O⁻, NH⁻ or S⁻;

Z is O or S;

each of R² and R³ is an alkyl group, H, a monovalent or divalent metal cationic salt, or an ammonium cationic salt, and R² and R³ may be the same or different.

Prodrugs of catechols can also be activated to the corresponding catechol in vivo by the action of non-specific phosphatases, sulphatases or other metabolic enzymes. The corresponding catechol will be oxidatively activated by an oxidizing agent or enzyme.

ii) Quinone Prodrugs. Since quinone drugs are highly unstable, conversion of a quinone to a corresponding prodrug form has the advantage of creating a stable molecule which is activated to regenerate the quinone in vivo by the action of non-specific phosphatases, sulphatases or other metabolic enzymes. Classes of drugs which contain the quinone moiety and which can be stabilized in phosphorylated prodrug form include:
1) Alkylating agents (Begleiter, Front. Biosci, 2000; Workman, Oncol. Res., 1994)-do not bind to DNA but intercalate into it resulting in changes in DNA replication. Anthracyclines such as Doxorubicin (Adriamycin), Mitomycin C, Porfiromycin, Diaziquone, Carbazilquinone, triaziquone, indoloquinone EO9, diaziridinyl-benzoquinone methyl DZQ, Anthracenediones, and Aziridines
2) DNA topoisomerase II inhibitors including Lapachones such as Beta-Lapachone (U.S. Pat. Nos. 5,969,163, 5,824,700, and 5,763,625)
3) Antibiotic compounds such as the Mitoxantrone, Actinomycin, Ansamycin benzoquinones and quinonoid derivatives including the Quinolones, Genistein, Bactacyclin,
4) Furanonapthoquinone derivatives and other naphthoquinones and naphtha-[2,3-d]-imidazole-4,9-dione compounds.

### Therapeutic Treatments

An object of the present invention is a method of producing an anti-tumor effect in a patient bearing a solid tumor comprising the administration of an effective amount of a quinone, catechol, or prodrug thereof. Anti-proliferative effects of a method of treatment of the present invention include but are not limited to: inhibition or delay of tumor cell growth or proliferation, or growth delay. These effects include tumor regression, tumor shrinkage, increased time to regrowth of tumor on cessation of treatment, and slowing of disease progression. It is expected that when a method of treatment of the present invention is administered to a patient in need of treatment for cancer, said method of treatment will produce an effect, as measured by, for example, one or more of: the extent of the anti-tumor effect, the response rate, the time to disease progression, and the survival rate.

In one embodiment, the compounds of the present invention may be used as antimicrotubule agents. Microtubules, cellular organelles present in all eukaryotic cells, are required for healthy, normal cellular activities. They are an essential component of the mitotic spindle needed for cell division, and are required for maintaining cell shape and other cellular activities such as motility, anchorage, transport between cellular organelles, extracellular secretory processes (Dustin, P. (1980) Sci. Am., 243: 66-76), as well as modulating the interactions of growth factors with cell surface receptors, and intracellular signal transduction. Furthermore, microtubules play a critical regulatory role in cell replication as both the c-mos oncogene and CDC-2-kinase, which regulate entry into mitosis, bind to and phosphorylate tubulin (Verde, F. et al. (1990) Nature, 343:233-238), and both the product of the tumor suppressor gene, p53, and the T-antigen of SV-40 bind tubulin in a ternary complex (Maxwell, S. A. et al. (1991) Cell Growth Differen., 2:115-127). Microtubules are not static, but are in dynamic equilibrium with their soluble protein subunits, the α- and β-tubulin heterodimers. Assembly under physiologic conditions requires guanosine triphosphate (GTP) and certain microtubule associated and organizing proteins as cofactors; on the other hand, high calcium and cold temperature cause depolymerization. Interference with this normal equilibrium between the microtubule and its subunits would therefore be expected to disrupt cell division and motility, as well as other activities dependent on microtubules.

When used as an anti-cancer agent, the compounds of the present invention can be formulated as a single composition or they may contain additional therapeutic agents, such as anti-cancer agents. Such therapeutic agents include, for example, a chemotherapeutic agent, an alkylating agent, a purine or pyrimidine analog, a vinca or vinca-like alkaloid, an etoposide or etoposide-like drug, an antibiotic, a corticosteroid, a nitrosourea, an antimetabolite, a platinum based cytotoxic drug, a hormonal antagonist, an anti-androgen, an anti-estrogen, or a derivative, modification or combination of these agents, and all other anti-cancer agents disclosed in this application.

In another aspect, the invention provides a method of treating a patient suffering from a vascular proliferative disorder comprising the administration of a quinone, catechol, or Prodrug in order selectively reduce the flow of blood in the proliferating vasculature of the patient. As used herein “Vascular proliferative disorders” includes any mammalian disease state in which the pathology of the disease is characterized by the presence of endothiulium, arteries, blood vessels, or neovasculature formed by undesirable and pathological angiogenesis that is associated with disease states. These include disease neoplastic and malignant disease states such as solid tumor cancer, as well as non-malignant disease states, including without limitation ocular diseases such as wet or age-related macular degeneration, diabetic retinopathy, retinopathy of prematurity, diabetic molecular edema, uveitis, and corneal neovascularization, and other disease states including psoriasis, rheumatoid arthritis, atheroma, restenosis, Kaposi's sarcoma, haemangioma, and, in general, inflammatory diseases characterized by vascular proliferation.

The catechol, quinone compounds of the present invention and their Prodrugs may be used as dual activity agents in order to generate an enhanced response in vascular proliferative disorders.

### Therapeutic Administration

Pharmaceutical compositions of the invention are formulated to be compatible with its intended route of administration. Pharmaceutical compositions may be prepared from the active ingredients or their salts in combination with pharmaceutically acceptable carriers.

As with the use of other chemotherapeutic drugs, the individual patient will be monitored in a manner deemed appropriate by the treating physician. Dosages can also be reduced if severe neutropenia or severe peripheral neuropathy occurs, or if a grade 2 or higher level of mucositis is observed, using the Common Toxicity Criteria of the National Cancer Institute.

The compositions of the present invention may also be formulated for systemic administration. Examples of systemic routes of administration include parenteral, e.g., intravenous, intradermal, subcutaneous, oral (e.g., inhalation), transmucosal, and rectal administration. Solutions or suspensions used for parenteral or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a vascular targeting agent) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from a pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In addition to the vascular targeting agents described above, the invention also includes the use of pharmaceutical compositions and formulations comprising a vascular targeting agent in association with a pharmaceutically acceptable carrier, diluent, or excipient, such as for example, but not limited to, water, glucose, lactose, hydroxypropyl methylcellulose, as well as other pharmaceutically acceptable carriers, diluents or excipients generally known in the art.

It is intended that the systemic and non-systemic administration of VTAs and tubulin binding agents in accordance with the present invention will be formulated for administration to mammals, particularly humans. However, the invention is not limited in this respect and formulations may be prepared according to veterinary guidelines for administration to animals as well.

Advantageously, the present invention also provides kits for use by a consumer for treating disease. The kits comprise a) a pharmaceutical composition comprising the claimed compounds and a pharmaceutically acceptable carrier, vehicle or diluent; and, optionally, b) instructions describing a method of using the pharmaceutical composition for treating the specific disease. The instructions may also indicate that the kit is for treating disease while substantially reducing the concomitant liability of adverse effects associated with antibiotic administration.

A “kit” as used in the instant application includes a container for containing the separate unit dosage forms such as a divided bottle or a divided foil packet. The container can be in any conventional shape or form as known in the art which is made of a pharmaceutically acceptable material, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. The container employed can depend on the exact dosage form involved, for example a conventional cardboard box would not generally be used to hold a liquid suspension. It is feasible that more than one container can be used together in a single package to market a single dosage form. For example, tablets may be contained in a bottle which is in turn contained within a box.

An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process, recesses are formed in the plastic foil. The recesses have the size and shape of individual tablets or capsules to be packed or may have the size and shape to accommodate multiple tablets and/or capsules to be packed. Next, the tablets or capsules are placed in the recesses accordingly and the sheet of relatively stiff material is sealed against the plastic foil at the face of the foil which is opposite from the direction in which the recesses were formed. As a result, the tablets or capsules are individually sealed or collectively sealed, as desired, in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It maybe desirable to provide a written memory aid, where the written memory aid is of the type containing information and/or instructions for the physician, pharmacist or subject, e.g., in the form of numbers next to the tablets or capsules whereby the numbers correspond with the days of the regimen which the tablets or capsules so specified should be ingested or a card which contains the same type of information. Another example of such a memory aid is a calendar printed on the card e.g., as follows “First Week, Monday, Tuesday,” . . . etc. . . . “Second Week, Monday, Tuesday, . . . ” etc. Other variations of memory aids will be readily apparent. A “daily dose” can be a single tablet or capsule or several tablets or capsules to be taken on a given day.

Another specific embodiment of a kit is a dispenser designed to dispense the daily doses one at a time. Preferably, the dispenser is equipped with a memory-aid, so as to further facilitate compliance with the regimen. An example of such a memory-aid is a mechanical counter, which indicates the number of daily doses that, has been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

In order to facilitate a further understanding of the invention, the following examples are presented primarily for the purpose of illustrating more specific details thereof. The scope of the invention should not be deemed limited by the examples, but encompass the entire subject matter defined in the claims. It will be apparent to those skilled in the art that many modifications, both to the materials and methods, may be practiced without departing from the purpose and interest of the invention.

### EXAMPLES

### Materials and Methods

Materials. CA1, CA1P and CA4 were obtained from Oxigene Inc.; solutions were freshly prepared each day and protected from light. PBS (0.14 M NaCl, 3 mM KCl, 10 mM phosphate) was from Oxoid Ltd. (Basingstoke, Hampshire, United Kingdom). HRP Type VIA, tyrosinase from mushroom, SOD from bovine erythrocytes and all other chemicals were obtained from Sigma (Poole, UK).

LC/MS. LCMS analyses were run on an Micromass Single Quadrupole LCMS system comprising an Agilent HP-1100 LC with a Hypersil BDS C₁₈ (5μ) reverse phase column (2.1×50 mm) run with a flow rate of 1.00 mL/min. The mobile phase used solvent A (H₂O/0.1% TFA) and solvent B (CH₃CN/0.1% TFA) with a 2.1 min gradient from 0% to 95% CH₃CN. The gradient was followed by a 0.2 min return to 0% CH₃CN and a 0.1 min flush. The peaks of interest eluted on the LC profiles at the times indicated.

Proton NMR. Unless otherwise indicated all ¹H NMR spectra were run on an Bruker Avance 400 MHz instrument. All observed protons are reported as parts per million (ppm) downfield from tetramethylsilane (TMS) or other internal reference in the appropriate solvent indicated.

Oxidation of CA1 to Q1. CA1 was dissolved in ethanol, and water added to give a final concentration of 0.8 mM in 1% ethanol; this was mixed with an equal volume of FeCl₃ (4 mM) in H₂SO₄ (1 mM). After 1 min, to prevent further oxidation to Q2, FeCl₃ was removed by solid phase extraction. The column (Discovery C18, 100 mg, 1 mL (Supelco, Poole, UK) was pre-conditioned with methanol followed by water, the sample loaded and the iron removed by washing with water followed by 20% methanol. The quinone (containing ˜50% unchanged CA1) was eluted with acetonitrile. Identity was confirmed by LC/MS and UV/vis absorbance. The mixture of quinone and CA1 was stored in the dark in the absence of water and was stable in solution throughout the day.

Oxidation of CA1 to Q2. CA1 was dissolved in ethanol, then water added to give a 2 mM solution containing 4% v/v ethanol, mixed with an equal volume of FeCl₃ (8 mM) in H₂SO₄ (2 mM) and stirred for 45 min before extracting in ethyl acetate. The product was pre-absorbed onto silica and purified through a silica column eluting with 3:1 hexane:ethyl acetate. The red fractions were collected and dried down on a rotary evaporator. Purity and identity was checked by HPLC comparing to previously recorded spectra with detection at 265 nm.

Oxidation of CA1 or CA4 by H₂O₂/Horseradish Peroxidase (HRP). Reaction rates of CA1 and CA4 with HRP compound I were measured by stopped-flow spectrophotometry as previously described (Candeias, L. P., et al. (1996) Biochemistry 35, 102-108). CA1 and CA4 were dissolved in ethanol and diluted with water to give a stock solution containing 4% v/v ethanol. Further dilution in phosphate buffer solution (10 mM, pH 7 or 7.4) gave a maximum ethanol concentration of 0.1% v/v. HRP compound I was formed by premixing (1 s) equimolar HRP and H₂O₂ (0.43 μM), and reaction monitored after adding CA1 or CA1 (0.1 to 2.5 μM) using double-mix conditions at 25° C. The formation of HRP compound II was monitored at 411 nm; five experiments were averaged and fitted to first-order (exponential) kinetics at five different concentrations of CA1 or CA4. Second-order rate constants were calculated from the linear fit of a plot of first-order rate constants against concentration.

Oxidation of CA1 or CA4 by Tyrosinase or Lactoperoxidase. Solutions of CA1 or CA4 (100 μM) were treated with mushroom tyrosinase (7.5 μg/mL, activity 2590 U/mg) at 37° C. in PBS, or with bovine erythrocyte lactoperoxidase (10 μg/mL, activity 5100 U/mg) and H₂O₂ (1 mM) with and without SOD (50 μg/mL, 4400 U/mg) at 28° C. in 25 mM phosphate buffer solution containing DTPA (100 μM). Reactions were monitored by HPLC.

Oxidation of CA1 by HL60 cells. Human pro-myelocytic leukaemia cells (HL-60) (European Collection of Cell Cultures, Salisbury, United Kingdom) were maintained in RPMI medium with 10% foetal calf serum, 2 mM L-glutamine, 100 units/mL penicillin and 100 μg/mL streptomycin. CA1 (87 μM) in PBS containing diethylenetriaminepentaacetic acid (DTPA, 100 μM) was mixed with HL-60 cells (2×10⁵) with or without the addition of SOD (125 μg/mL) at 37° C. The loss of CA1 and formation of Q2 were measured by HPLC.

HPLC analysis of CA1, Q1 and Q2. HPLC was carried out using a gradient of 10 mM ammonium formate containing 20% methanol (A) and methanol (B), 30-100% (B) over 3 min at 1 mL/min. A Hichrom RPB column (100×3.2 mm) column was used with detection by UV-visible absorbance (Waters 2996) and electrospray mass spectrometry (Waters Micromass ZQ) operating in ES+mode at 2.5 kV with a cone voltage of 20-22 V. Q2 was chromatographed on an ACE C18 column (125×3 mm) using a gradient of 10 mM ammonium formate (A) and acetonitrile (B), 30-60% (B) over 5 min at 1 mL/min.

Pharmacokinetics and Metabolism of CA1P/CA1 in Mice. CA1P in water (5 mg/mL) was injected IP into CBA female mice with a subcutaneous dorsal CaNT tumour. After 15 min-2 h, duplicate mice were sacrificed and blood and tissues removed. Blood was collected into tubes containing 1 mg K₃EDTA and 1 mg ascorbic acid, centrifuged (14,000 g, 2 min) and the plasma stored at −20° C. Liver, tumour and kidney samples were removed and homogenized in 4 vol 2 mg/mL Na₂EDTA/1 mg/mL ascorbic acid, and stored at −20° C. before analysis. For CA1 analysis, 50 μL of plasma or homogenate was extracted in an equal volume of ˜3 mg/mL desferroxamine mesylate suspended in acetonitrile, and the supernatant injected directly into the HPLC. Samples were chromatographed on a Hichrom RPB column (100×3.2 mm) eluted isocratically with 38% acetonitrile, 75 mM HClO₄, 5 mM KH₂PO₄, pH 2.65 at 0.6 mL/min with a column temperature of 35° C. A Coulochem 5100A electrochemical detector with a porous graphite electrode was used, operating at +0.35V. To confirm Q1H₂—SG formation in vivo, non-tumour bearing SCID male mice were treated as described above along with a non-treated control and sacrificed after 18 min. Tissue homogenates were extracted with equal volumes of acetonitrile, centrifuged and the supernatant dried down under N₂. Samples were reconstituted in 80 μL 20% methanol in 10 mM sodium formate and chromatographed on a Hichrom RPB column (100×3.2 mm) with a gradient of 20% methanol in 10 mM ammonium formate changing to 100% methanol, in 5 min at 1 mL/min. Detection using mass spectrometry in ES+ single ion mode (m/z 638) was used (cone voltage 15 V) with samples compared to prepared Q1H₂—SG.

Measurement of Oxygen Consumption by Q2 with Antioxidants. Q2 was dissolved in acetonitrile, and phosphate buffer (25 mM, pH 7.43) containing DTPA (100 μM) added to give a 50 μM solution in 1% v/v acetonitrile; a 3 mL sample was added to a stirred Clark-type oxygen electrode chamber without headspace (Rank Brothers, Cambridge, United Kingdom) at 37° C. Ascorbate (50 μL, 18 mM) or glutathione (100 μL, 90 mM) was added when the signal stabilized to give final concentrations of ˜0.3 and 3 mM respectively, and oxygen consumption recorded.

Measurement of oxygen consumption by CA1/HRP/H₂O₂ with ascorbate. CA1 (100 μM) and H₂O₂ (10 μM) (3 mL) in the oxygen electrode chamber at 37° C. was mixed with HRP (6.7 μg/mL) for 0, 1, 3 or 10 min before the addition of ascorbate (1 mM final concentration), and oxygen consumption recorded.

EPR Spectroscopy. A Bruker EMX spectrometer (Bruker, Coventry, United Kingdom) equipped with a high sensitivity cylindrical cavity was used with 100 kHz modulation frequency. Typical spectrometer settings were: modulation amplitude, 0.025 mT (0.1 mT for DMPO experiments); microwave power, 20 mW; sweep rate, 0.024 mT/s (0.2 mT/s for DMPO experiments); time constant, 20 ms (10 ms for DMPO); gain, 2-4×10⁵ (4-20 sweeps averaged). All experiments utilized phosphate buffer (0.2 M, pH 7.6) treated with Chelex 100, 5,5-dimethyl-1-pyrolline-N-oxide (DMPO, 0.1 M), ethanol (10% v/v) and DTPA (2 mM).

Pulse Radiolysis. The apparatus for irradiating solutions with ˜0.5 μs pulses of ˜6 MeV electrons and monitoring reactions by kinetic spectrophotometry with sub-microsecond resolution has been described (Candeias, L. P., et al. (1994) J. Phys. Chem. 98, 10131-10137). The interaction of the hydroquinone with azide radicals was measured by pulse radiolysis (6 Gy) of solutions containing CA1 (38 μM) with sodium azide (50 mM) in sodium phosphate buffer (10 mM) at pH 7.35, saturated with either N₂O or N₂O/O₂ 80/20 v/v. Radical spectra were measured by calculating the radical extinction coefficient of CA1 or CA4 (50 μM containing 0.05% v/v DMSO) with sodium azide (0.1 M) (4.5 Gy/pulse) at varying wavelengths (240-650 nm). Reduction of Q2 by O₂.⁻ or CO₂.⁻ was studied by dissolving Q2 in acetonitrile, and diluted with sodium formate (0.1 M) in sodium phosphate buffer (25 mM, pH 7.4) to give a solution of Q2 (30 μM) in 1% (v/v) acetonitrile/H₂O. Solutions were saturated with O₂ or N₂ before radiolysis (3 Gy/pulse) and the semiquinone spectrum recorded after 200 μs.

Cyclic Voltammetry. Voltammograms were recorded with an Autolab PGSTAT 20 potentiostat with GPES software (Windsor Scientific, Slough, UK). The three-electrode system consisted of a working disk (3 mm) glassy carbon electrode (GC), a reference saturated calomel electrode (SCE) and a platinum wire auxiliary electrode. The reference electrode potential was checked against chemical standard grade potassium hexachloroiridate (IV) (K₂IrCl₆). Before a scan, the working electrode was polished with alumina slurry (0.05 μm), and rinsed with water. CA1 was dissolved in DMSO, or Q2 in acetonitrile and diluted to provide solutions containing CA1 or Q2 (0.5 mM), supporting electrolyte (KCl) (0.1 M) and phosphate buffer (5-25 mM) in 0.5% v/v DMSO/H₂O (CA1) or 10% v/v acetonitrile/H₂O (Q2). To vary the pH, small amounts of concentrated HClO₄ or NaOH were added to the working solution. The solutions were bubbled with N₂ and the sweep rate was 0.1 V/s (CA1) or 0.1-1 V/s (Q2). All experiments were carried out at room temperature (24±2° C.).

### Example 1

### Identification of Quinones Produced on Oxidation of CA1

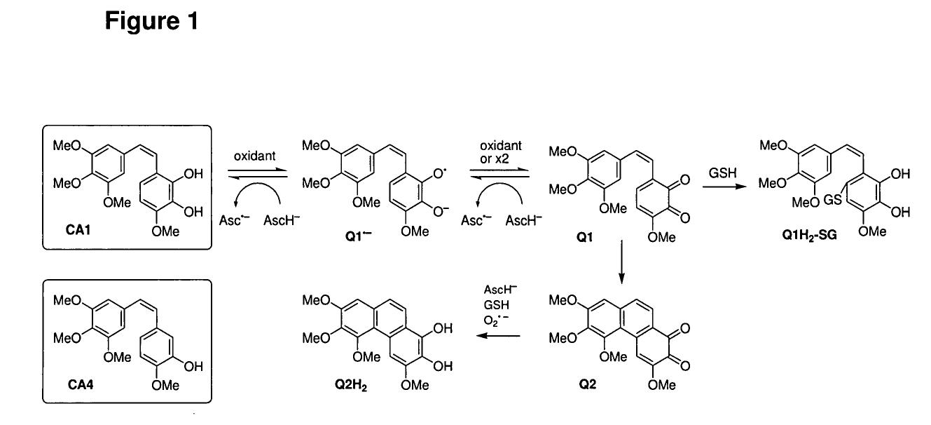

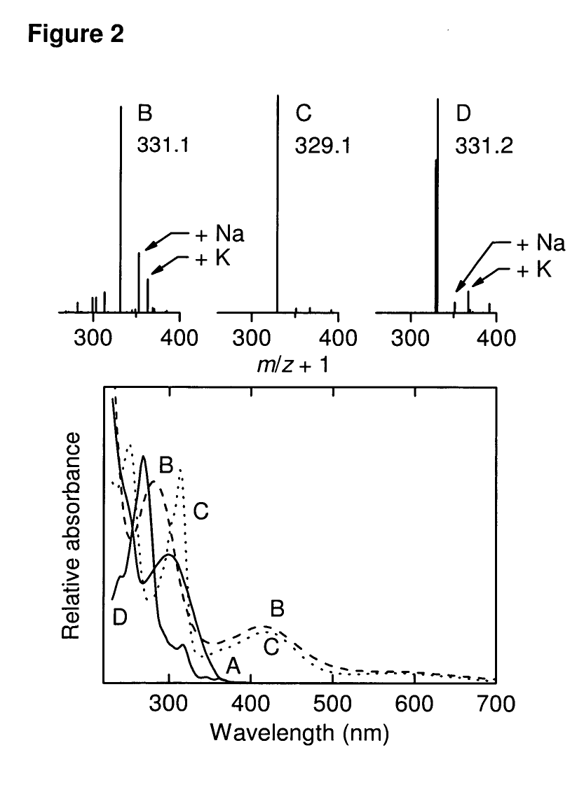

CA1 (λₘₐₓ 300 nm, FIG. 2(A), mass ˜332 Da) was initially oxidized by FeCl₃ (1 min oxidation with immediate removal of FeCl₃, with only ˜50% loss of CA1, see Materials and Methods) to yield a compound with λₘₐₓ 280 and 412 nm and mass ˜330 Da (FIG. 2(B)). This is consistent with oxidation of the catechol to the corresponding quinone Q1 (loss of two hydrogen atoms) (FIG. 1). The same product, with absorption maxima at 312 and 412 nm and identical HPLC retention time and mass spectral pattern, was also initially produced on oxidation of CA1 by HRP compound I (see below), lactoperoxidase, tyrosinase, or HL60 cells (in the presence of SOD) (data not shown).

Q1 was unstable in aqueous solution, resulting in the formation of a more hydrophobic product absorbing at 312 and 412 nm and with mass of ˜328 Da (FIG. 2(C)), consistent with the formation of a phenanthrene quinine product (Q2) resulting from electrocyclic ring closure (FIG. 1). Under the same HPLC conditions, no similarly-retained products were formed on oxidation of CA4.

In order to support the identity of Q2 it was synthesised by oxidation of CA1 with FeCl₃/H₂SO₄ and analysed by accurate mass measurement, and ¹H and ¹³C NMR. M/z (ES⁺): 329.1 (MH⁺, 100%) and 679.2 (40%); calculated C₁₈H₁₇O₆: 329.1025, observed: 329.1038. ¹H NMR (CDCl₃, D₂O, 500 MHz): δ_{H} 8.43 (1H, s, ArCH), 7.91 (1H, d, J=8.5 Hz, ArCH), 7.55 (1H, d, J=8.5 Hz, ArCH), 6.91 (1H, s, ArCH), 4.02 (3H, s, OMe), 3.99 (3H, s, OMe), 3.95 (3H, s, OMe) and 3.91 (3H, s, OMe). ¹³C NMR (CDCl₃): δ_{C} 178.9 (C═O), 176.3 (C═O), 155.4, 151.7, 151.1, 144.2, 136.6, 133.4, 127.3, 125.6, 124.9, 120.0, 114.2, 104.4, (C—H), 65.8, 61.7, 56.0 and 55.5 (CH₃). IR (KBr): 1678 cm⁻¹ (Ar—C═O).

### Example 2

### Reaction of CA1 Quinones Q1 and Q2 with Glutathione

Adding GSH to Q1 resulted in immediate decoloration and formation of a polar, stable product (FIG. 3, peak 4) with a mass ˜637 Da, consistent with the formation of a quinone-glutathione adduct Q1H₂—SG (FIG. 1). FIG. 1 shows GSH adding to the position of the more electropositive of the positions potentially susceptible to Michael addition, although this has not been confirmed.

Q2 was prepared from CA1 by oxidation with FeCl₃ as described above, with 99% purity, and excess GSH added. Chromatographic analysis showed loss of Q2 and formation over several minutes of a similarly-retained but slightly more polar peak with λₘₐₓ 270 nm and mass of ˜330 Da (FIG. 2(D)) suggestive of reduction of Q2 to a hydroquinone Q2H₂ (FIG. 1). No evidence of a thiol conjugate was seen with Q2 and GSH.

### Example 3

### Tissue Distribution and Metabolism of CA1 after Administration to Mice

Free CA1 was found to be retained in mouse CaNT tumor tissue (9.2 μM) compared to plasma (0.085 μM) and liver (2.0 μM) 2 h after IP injection of CA1P (50 mg/kg). A metabolite with HPLC retention characteristics and MS fragmentation patterns identical to that of Q1H₂—SG was observed in all tissues, with the highest levels found in the liver 15 min after dosing (FIG. 4). The same product (mass ˜637 Da) was measured in the liver of non-tumor bearing SCID mice after CA1 administration (FIG. 2), and in low amounts in plasma; no peak attributable to Q1H₂—SG was observed in kidney homogenates.

### Example 4

### Reactions of CA1 Quinones Q1 and Q2 with Ascorbate

Addition of excess ascorbate to Q1 showed immediate loss of Q1 with the re-formation of CA1 (FIG. 3). FIG. 1 shows this as proceeding via two one-electron steps, on the basis of EPR evidence for the ascorbate radical Asc.⁻ (see below).

### Example 5

### Oxygen Consumption During Reaction of CA1 Quinones with GSH or Ascorbate

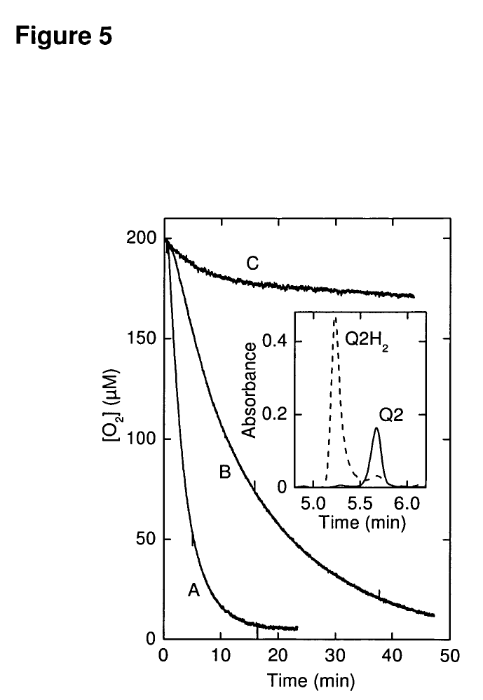

Measurements using an oxygen electrode (FIG. 5) showed that the oxygen concentration in air-saturated buffer containing Q2 at 37° C. was rapidly reduced on adding either ascorbate or GSH. Catalase (10 μg/mL) was not found to affect the rate of oxygen loss. HPLC analysis of the solutions after O₂ consumption was complete (FIG. 5, insert) showed the formation of a product with the same UV spectrum and mass (˜330 Da) as seen in the previous LC/MS experiments and ascribed to Q2H₂ (FIG. 3(D)).

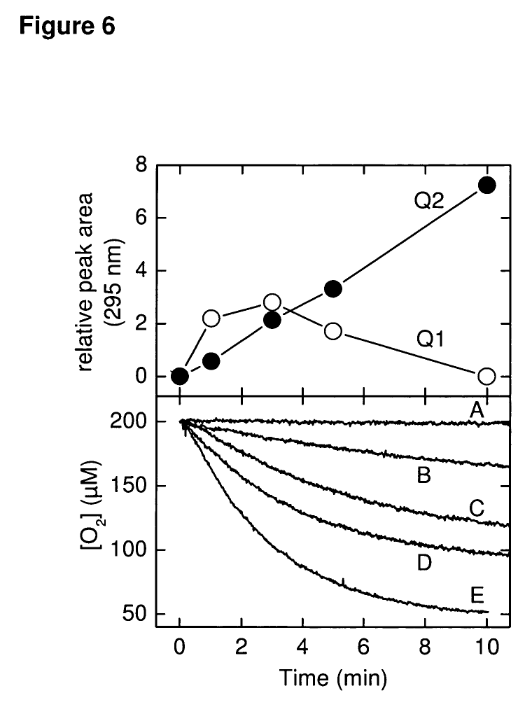

Peroxidase-catalysed oxidation of CA1 by HRP/H₂O₂ resulted in formation of Q1, which decayed over a few minutes leaving Q2 (FIG. 6(A)). There was no oxygen consumption if ascorbate was added to a mixture of CA1 and HRP/H₂O₂ immediately on mixing CA1 with HRP/H₂O₂; however, if the addition of AscH⁻ was delayed by a few minutes, thus facilitating build-up of Q2, O₂ was consumed in a delay-time-dependent manner (FIG. 6(B)).

### Example 6

### Production of Free Radicals During Oxidation of CA1 or CA4 and the Effects of GSH or Ascorbate

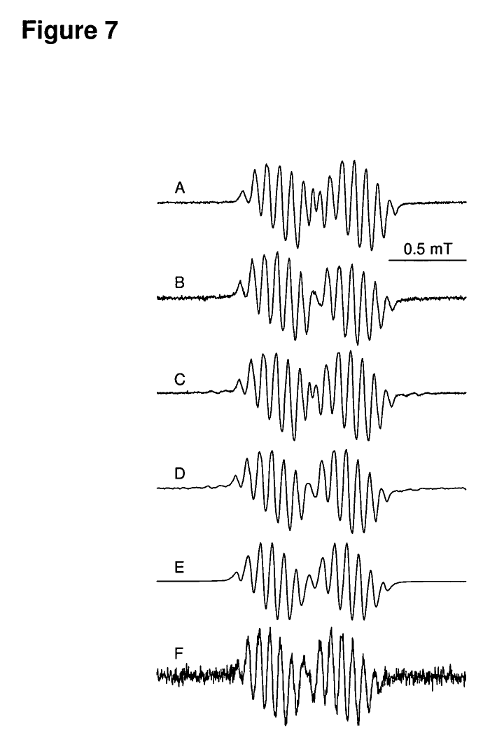

EPR signals were observed in aqueous solutions of CA1 at pH 7.4 (FIG. 7). The signals were enhanced by adding MgCl₂, which stabilizes catechol semiquinone radicals (Kalyanaraman, B., et al. (1987). J. Biol. Chem. 262, 11080-11087). FIG. 7(A-C) shows similar signals were obtained from CA1 alone (autoxidation), or with added tyrosinase or HRP. Although in this experiment, signal (B) was ˜40% lower in intensity than signal (A), signal intensities reflected time standing in air as much as added enzyme, and increase in signal intensity on adding either enzyme were never greater than 20% higher than without enzyme. Weak outer lines were evident in spectrum (C), not seen in (A). A similar signal (D) was observed on dissolving the oxidized product Q2 in MgCl₂ buffer. Analysis of spectra (A-D) showed satisfactory simulations for three interacting protons together with the three protons of a methoxy substituent, with proton hyperfine splittings of a_{H}=0.479-0.482 mT, 0.150-0.155 mT, 0.080-0.082 mT (three equivalent), and 0.061-0.075 mT; simulation (D) represents the mean values. Omitting MgCl₂ resulted in a ˜4-fold weaker signal of overall similar pattern (E) but with mainly slightly-reduced couplings: a_{H}=0.465, 0.133, 0.068 (three equivalent) and 0.085 mT. Under similar conditions no signals were observed from CA4.

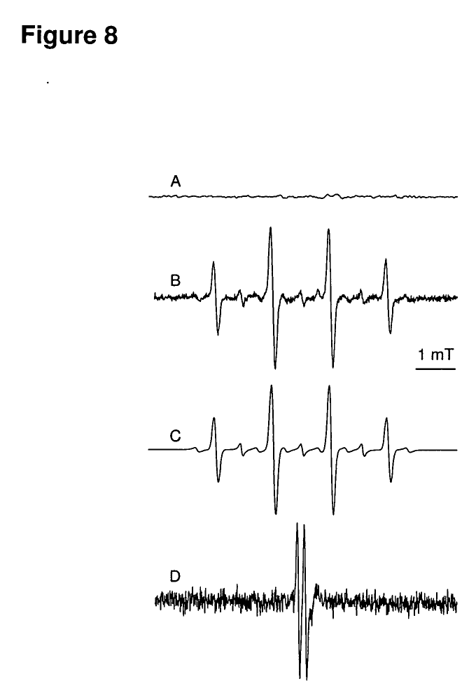

Because adding GSH or ascorbate to Q2 resulted in oxygen depletion (see above), intermediate radicals were identified using the spin trap DMPO. Adding GSH to Q2 in the presence of DMPO gave the characteristic four-line signal from DMPO/.OH, plus weak contributions from other species (FIG. 8(B); no signal was seen without Q2 (trace (A)). Simulation (FIG. 8(C)) showed a satisfactory fit assuming 75% of the signal came from DMPO/.OH (species 1), with 15% from a second species and 10% from a third, with the couplings indicated. On substituting GSH with ascorbate, these signals disappeared and only the doublet (a_{H}=0.179 mT) of the ascorbate radical was observed. The latter radical is always present in solutions containing ascorbate, but its intensity was approximately trebled by the addition of Q2.

### Example 7

### UV/Vis Spectra of the Semiquinone Radicals from CA1 and their Reactivity Towards Oxygen

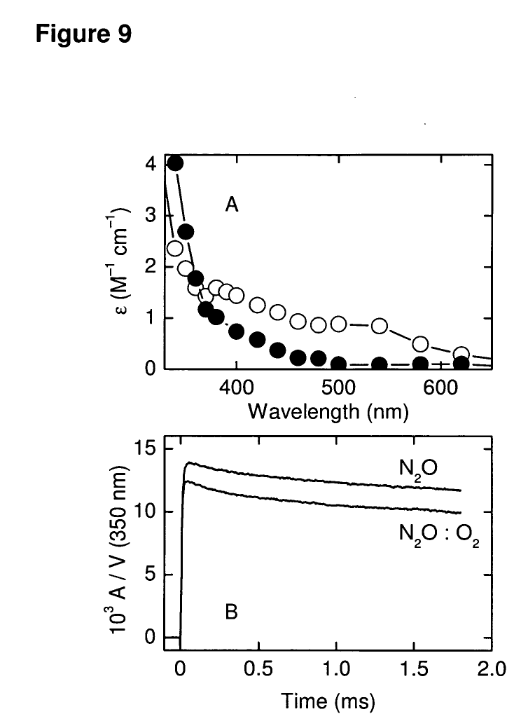

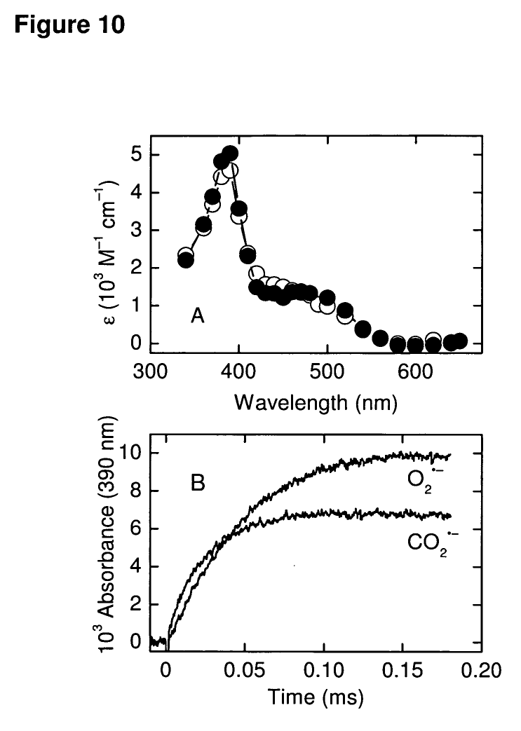

Pulse radiolysis was used to characterize the spectra of the radicals obtained either on one-electron oxidation of CA1 (Q1.⁻, FIG. 9(A)) or on one-electron reduction of Q2 (Q2.⁻, FIG. 10(A)); FIG. 9(A) also shows the spectrum of the radical obtained on oxidizing CA4 by N₃. The absorbance change, and stability of the transient species, from oxidation of CA1 was similar in the absence and presence of oxygen (FIG. 9(B)); oxidation of CA1 by the one-electron oxidant N₃. occurred with a rate constant of 4.9×10⁹ M⁻¹ s⁻¹ (data not shown). FIG. 10(A) shows that both the powerful reductant, CO₂.⁻, and the much weaker reductant, O₂.⁻, reacted with Q2 to produce a transient radical with spectra which were closely similar. Reduction by O₂.⁻, unlike the case with CO₂.⁻, necessarily involves solutions containing O₂, but the transient radical produced was unreactive towards oxygen, at least over hundreds of microseconds (FIG. 10(B)).

### Example 8

### Comparison of Rates of Oxidation of CA1 and CA4 by Enzymes, and Oxidation of CA1 in Cells

CA1 was found to be oxidized by HRP compound I, an oxidizing peroxidase intermediate (Dunford, H. B. (1999) Heme Peroxidases, Wiley-VCH, New York), with formation of HRP compound II. Second order rate constants of 7.7±0.2×10⁶ (pH 7) or 9.0±0.2×10⁶ M⁻¹ s⁻¹ (pH 7.4) were measured; similar experiments with HRP compound I and CA4 yielded rate constants of 3.4±0.2×10⁷ (pH 7) or 5.1±0.1×10⁷ M⁻¹ s⁻¹ (pH 7.4). Lactoperoxidase and tyrosinase were also effective in oxidising CA1 (100 μM) with up to 70 μM loss of CA1 in 80 min at 37° C. In comparison 20 μM CA4 (100 μM) was lost in the same conditions (see Materials and Methods). SOD had little effect on purified enzyme turnover.

HL-60 (human promyelocytic leukemia) cells are rich in myeloperoxidase, but while CA1 (87 μM) was oxidized slowly by air at pH 7.4, 37° C. (7 μM lost in 60 min), this was not detectably accelerated on adding HL-60 cells (˜2×10⁵ cells/mL). However, adding SOD (125 μg/mL) markedly accelerated CA1 loss in the presence of HL60 cells (37 μM loss in 60 min).

### Example 9

### Redox Properties of CA1, CA4 and Q2 Measured by Cyclic Voltammetry

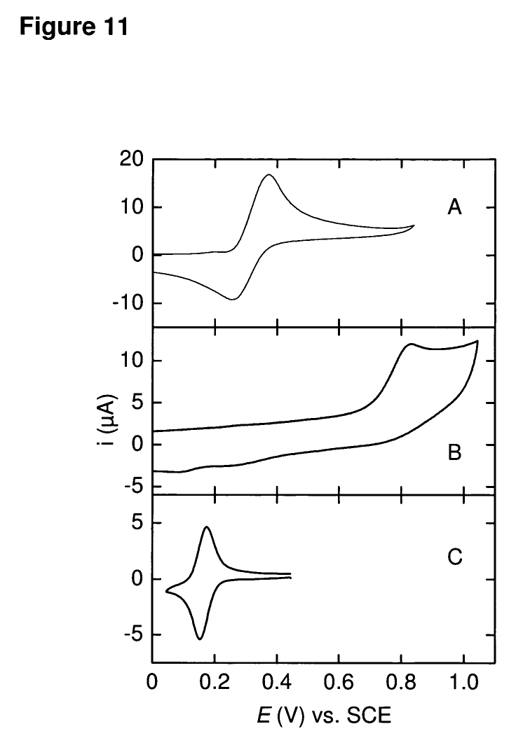

Cyclic voltammetry experiments confirmed major differences in the redox properties of CA1 and CA4. While CA1 was oxidized at potentials<0.4 V vs. NHE, simulation suggesting a mixture of one- and two-electron reversible reactions and a reduction potential for the CA1 radical (QH./QH₂) of ˜0.31 V at pH 7.34 (FIG. 11(A)), CA4 was oxidized at much higher potentials, with a non-reversible wave at 0.85 V vs. NHE (FIG. 11(B)). Cyclic voltammetry experiments with Q2 showed a one-electron reversible reaction with a reduction potential (Q2/Q2.⁻) of 0.16 V vs. NHE (FIG. 11(C)).

### Example 10

### Alternative Synthesis of Tricyclic Quinones

Quinone compounds of the invention may be prepared synthetically from the corresponding catechol by oxidation with o-chloranil in diethyl ether. A representative scheme is provided below.

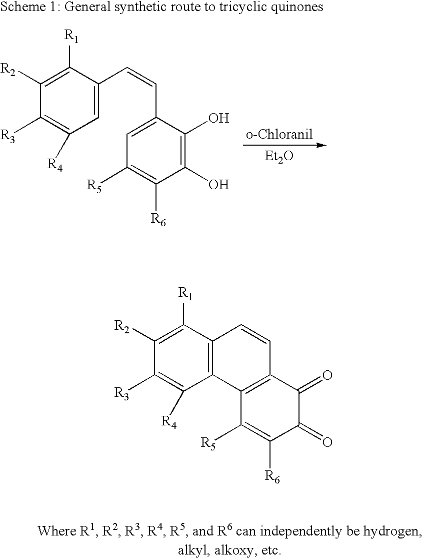

### a) Synthesis of CA1 Phenanthraquinone, 6 (Q2)

The phenanthraquinone analog of CA1 was synthesized using the oxidant O-chloranil.

To a solution of Combretastatin A-1 (0.050 g, 0.15 mmol) in Et₂O (1 ml) was added O-chloranil (tetrachloro-1,2-benzoquinone, 0.037 g, 0.15 mmol) with stirring for ½ hr. The reaction turned dark red in color. Reaction was followed by TLC until no starting material was left. The dark colored solid product obtained in quantitative yield was filtered and washed with hexanes and small amounts of ice cold ether.

6, ¹H NMR: in CDCl₃ δ (PPM) 8.43 (s, 1H, Ar—H), 7.93 (d, 1H, J=8.6 Hz, Ar—H), 7.53 (d, 1H, J=8.1 Hz, Ar—H), 7.26 (s, 1H, Ar—H), 6.91 (s, 1H, Ar—H), 4.02 (s, 3H, —OCH₃), 4.01 (s, 3H, —OCH₃), 3.98 (s, 3H, —OCH₃), 3.92 (s, 3H, —OCH₃).

¹³C NMR: in CDCl₃ δ (PPM) 178.92, 176.27, 155.46, 151.69, 151.10, 144.26, 136.64, 133.39, 127.33, 125.61, 124.88, 120.03, 114.19, 104.43, 61.74, 61.43, 56.05, 55.54.

### Example 11

### Alternative Synthesis of Tricyclic CA1 Catechols

Catechol compounds may be prepared synthetically by a Wittig reaction between an appropriately substituted aldehyde and an appropriately substituted phosphorous ylide. The aldehyde portion and ylide portion can be readily switched as well to allow for the judicious incorporation of the requisite functional groups within the target stilbenes (see Scheme 2 for general synthetic protocols).

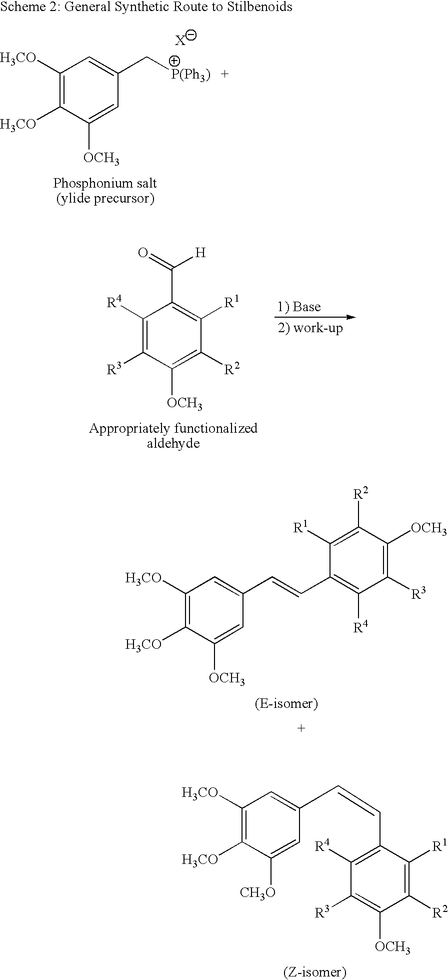

In certain aspects, tricyclic catechol compounds of the invention may be prepared synthetically from the bis-TBS protected stillbene catechol. For example, the bis-protected catechol is taken up in solution with iodine and irradiated to give the desired tricyclic bis-TBS protected catechol, in a procedure adapted from Singh et al., J. Org. Chem., 1989, 54, 4105. Deprotection with TBAF in THF yields the desired un-protected catechol.

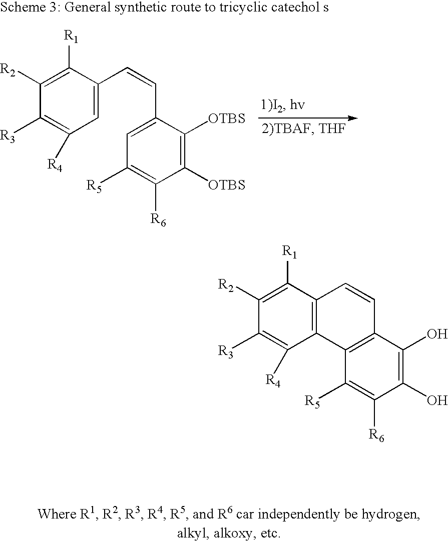

### Example 12

### Evaluating Therapeutic Properties

### a) Tubulin Binding Activity

The method of Verdier-Pinard (1998, Molec. Pharmacol. 53, 62-76) may be used to assay tricyclic catechol of the invention compounds for inhibition of tubulin polymerization. Tubulin polymerization is followed turbidimetrically at 350 nm on an Agilent 8453 spectrophotometer equipped with a kinetics program, a jacketed cell holder, and two microprocessor-controlled water baths. Purified tubulin (1 mg/ml) is induced to polymerize in a monosodium glutamate/GTP solution by a jump in temperature. Absorbance is recorded every 10 seconds and the data analyzed by a GraphPad Prism program.

### b) Tumor Cell Cytotoxicity

Exponentially growing tumor cells are treated with a compound of the invention for 24 hours. Insoluble compounds are formulated in a small amount (0.3%) of DMSO for biological evaluation. Cell viability is determined by the calorimetric MTT assay using 3(4,5-dimethylthiazol-2-yl)-2,5-diphenyl-2H-tetrazolium bromide according to well-established procedures (see Berridge, et al. (1996) for a general protocol of this type of assay).

### c) Reduction in Tumor Blood Flow

Compounds of the invention are dissolved in 50% DMSO (2 mg/kg) prior to intravenous (iv) administration (i.v.) to tumor-bearing mice. MHEC-5T tumors are established by subcutaneous injection of 0.5×10⁶ cultured MHEC5-T cells (German Collection of Microorganisms and Cell Culture, Braunschweig, Germany) into the right flank of Fox Chase CB-17 severe combined immunodeficient (SCID) mice. Tumor grafts grow palpable within one week and reached the limited size (15×15 mm) within 10 days. Tumor bearing mice are injected intraperitoneally with saline control or various dosages of a compound of the invention after the transplanted tumors reached a size of 300 mm³ (a size without development of necrosis). Twenty-four hours later they are injected with 0.25 ml of fluorescent FluoSphere beads (0.1 μm beads conjugated with blue fluorescent tag (F-8789, Molecular Probes, Eugene, Oreg.) and diluted 1:6 in physiological saline) in the tail vein, and sacrificed after 3 minutes. Tumors are then excised for cryosections. Cryosections of 8 μm thickness are directly examined under a fluorescent microscope. Functional blood vessels were indicated by blue fluorescence from injected microbeads. For quantification, three sections from three tumors treated in each group are examined and in each section, more than 70% of the area is automatically recorded with a microscopic digital camera at ×10 magnification. A computer program named Stage Pro (Media Cybernetics, MD) is used to control the picture recording.

Image analysis was performed with Image Plus software (Media Cybernetics, MD). The results are expressed as vessel area per mm² in percentage of the control.

### Discussion

Combretastatin A-1 was found to be retained in murine tumours relative to plasma or liver as shown previously (Kirwan, I. G., et al. (2004) Clin. Cancer Res. 10, 1446-1453), presumably because of tumour vascular shut down trapping CA1 inside the tumour vessels. Oxidation of CA1 by Fe(III) formed two products, thought to be ortho quinones Q1 and Q2 (FIG. 1); Q2 was fully characterized, while HPLC/MS provided support for the assignment to Q1. Further support for two different quinones was obtained by the different absorption spectra of radicals obtained on oxidation of CA1 (FIG. 2(A), assigned to Q1.⁻) or reduction of chemically-synthesized Q2 (FIG. 3(A), assigned to Q2.⁻). While both Q1 and Q2 reacted with GSH, the former generated a thioether adduct Q1H₂—SG, whilst Q2 produced another catechol, Q2H₂. Although ascorbate served to reverse oxidation of both quinones, reactivity of Q1 towards GSH must be sufficiently high to outweigh the competing reaction with ascorbate, since Q1H₂—SG was detected in the liver, tumor and plasma of mice administered with CA1P. High reactivities towards GSH and ascorbate of 4-methoxy-1,2-benzoquinone have been reported (Land, E. J., et al. (1990) Biochem. Pharmacol. 39, 1133-1135). The transformation of Q1 to Q1H₂—SG in vivo is likely to be catalysed by glutathione-S-transferases (even fast conjugative reactions of GSH are substrates for this enzyme family (Coles, B., (1988) Arch. Biochem. Biophys. 264, 253-280). This data enclosed herein has thus revealed Q1H₂—SG as a possible marker for oxidative metabolism of CA-1, via quinone formation and reaction with GSH. It is possible, like other GSH conjugates (Glutathione Conjugation. Mechanisms and Biological Significance; Sies, H., and Ketterer, B., Eds.; Academic Press: London, 1988), the thioether is degraded further to mercapturic acids.

In the absence of GSH, or possibly in parallel, the quinone Q1 formed initially is transformed rapidly to a closed-ring quinone Q2 or as enzyme-catalysed oxidation proceeds (FIG. 5(A)). Electrocyclic ring closure of Q1 leaves only one carbon atom free to undergo Michael addition with GSH, whereas there are two possible sites in Q1. The methoxy group adjacent to the free carbon in Q2 will increase electronegativity at this site, rendering it less likely to be attacked by a nucleophile than the site suggested for GSH attack on Q1 in Scheme 1; there may also be less steric hindrance for attack by GSH in Q1 compared to Q2. Thus ascorbate may be a potentially more important reductant than GSH for Q2, and the closed-ring hydroquinone Q2H₂ an alternative, though possibly less abundant, marker of oxidative metabolism of CA1 than Q1H₂—SG. An earlier study (Kirwan, ibid) suggested formation of a quinone from CA1, with mass corresponding to Q2, but the product isolated from mouse plasma and suggested to be a quinone seems unlikely to be the same as either Q1 or Q2 in the present study: the HPLC conditions and the peak showed mass fragments as high as 451.2.

Peroxidases are obvious candidates for catalysing oxidation of CA-1 in vivo. The widely-studied plant peroxidase, HRP, was shown to catalyse formation of Q1, while CA1 was shown to be a substrate for the mammalian peroxidase, lactoperoxidase. Myeloperoxidase-rich HL-60 cells oxidized CA-1 provided extracellular SOD was added. Macrophage infiltration of tumors may be a significant source of peroxidases. Phenols are good substrates for peroxidases, and higher reactivity of resorcinol (1,3-dihydroxybenzene) compared to phenol for oxidation by HRP Compound I is consistent with established redox relationships (Job, D., and Dunford, H. B. (1976) Eur. J. Biochem. 66, 607-614; Candeias, L. P., et al. (1997) Biochemistry 36, 7081-7085). While the reactivity of both CA1 and CA4 towards HRP compound I is comparable to that for reaction of compound I with other phenols (Candeias, ibid), the expected enhancement of reactivity accompanying the additional hydroxyl substituent in CA1 was not observed despite the much greater ease of oxidation of CA1 compared to CA4 demonstrated by cyclic voltammetry. A speculative explanation for this behaviour might be that oxidation of both combretastatins by HRP involves electron transfer from the trimethoxybenzene moiety to form a radical-cation that deprotonates at phenolic oxygen to form phenoxyl radicals.

Most peroxidases oxidize phenolic substrates via two one-electron steps (Dunford et al, ibid), i.e. producing phenoxyl radicals, or in the case of catechols, semiquinones. However, tyrosinase is thought to produce semiquinone radicals with catechols via ‘reverse disproportionation’ (Mason, H. S., et al. (1961) Biochem. Biophys. Res. Commun. 4, 236-238), where QH₂ is the catechol and Q the corresponding quinone:

QH₂+Q→2Q.⁻+2H⁺. (1)

Reductive addition of GSH to quinones, such as that forming the thioether hydroquinone Q1H₂—SG (FIG. 1), can also generate radicals via similar equilibria (Gant, T. W., et al. (1986) FEBS Lett. 201, 296-300; Takahashi, N., et al. (1987). Arch. Biochem. Biophys. 252, 41-48). Partial aerobic oxidation of catechols, especially at alkaline pH, is sufficient to generate enough quinone such that semiquinones are readily observed in aqueous solutions, with EPR signals enhanced by complexing the semiquinones with Mg²⁺ or Zn²⁺ (29).

It was hypothesized that the EPR signals observed on oxidation of CA1 (FIG. 7) might provide evidence for the semiquinones of either Q1 or Q2, although a mixture of both might be formed and the signal reflects the steady-state situation. While small differences were apparent in the different experiments, the signal obtained from Q2 alone in the presence of Mg²⁺ (perhaps via reduction by metal ion contaminants) was similar to that obtained via CA1, suggesting at first sight that most of the signal observed arose from Q2 semiquinone. However, the dominant features (simulation, FIG. 7(E) are three proton splittings with a_{H}˜0.48, 0.15 and 0.07 mT, and methoxy proton couplings of ˜0.08 mT; these parameters are rather similar to that reported from the semiquinone of 3-methoxycatechol, which has a_{H}˜0.48 (H-5), 0.125 (H-4), 0.059 mT (H-6), and 0.067 mT (OCH₃) (Holton, D. M., and Murphy, D. (1982) Journal of the Chemical Society, Faraday Transactions 178, 1223-1236; Steenken, S., and O'Neill, P. (1977) J. Phys. Chem. 81, 505-508). (The semiquinone of Zn²⁺-complexed 4-methoxycatechol shows the major proton hyperfine couplings only ˜8-17% higher than that of the uncomplexed radical (Kalyanaraman, B., et al., (1985) Environ. Health Perspect. 64, 185-198). While the H-6 splitting in 3-methoxycatechol will not, of course, be a feature in Q1.⁻ (and couplings analogous to both H-5 and H-6 protons in 3-methoxycatechol are unavailable in Q2.⁻), the dominant H-5 splitting in 3-methoxycatechol semiquinone, a model for Q1.⁻ (a_{H}˜0.48 mT (Holten et al., ibid; Steenken et al., ibid), is similar to that of H-4 in 1,2-dihydroxynaphthalene semiquinone, a model for Q2.⁻ (˜0.45 mT (Ashworth, P., and Dixon, W. T. (1974) J. Chem. Soc., Perkin Trans. 2, 739-744). Likely proton couplings for the exocyclic, vinylogous protons in Q1.⁻ are difficult to estimate, but comparison with the methyl protons in 3,4-dimethoxy-6-methylcatechol (a_{H}=0.185 mT (Holton, D. M., and Murphy, D. (1980) J. Chem. Soc., Perkin Trans. 2, 1757-1759)) or the exocyclic proton couplings in caffeic acid (3,4-dihydroxycinnamic acid) (˜0.24 and 0.12 mT (Ashworth, P. (1976), J. Org. Chem. 41, 2920-2924; Bors, W., et al., (2003) Biochim. Biophys. Acta 1620, 97-107)) suggests the larger exocylic proton coupling in Q1.⁻ might be fairly similar to the corresponding coupling in Q2.⁻. Thus the protons on the formerly vinylogous substituent exocyclic to the catechol moiety in Q2.⁻ might have a larger coupling not dissimilar to H-8 of the semiquinone of 1,2-dihydroxynaphthalene, which has a_{H}=0.13 mT (Ashworth et al., ibid). Hence it is difficult from the EPR parameters to assign the dominant signal unequivocally to Q1.⁻ or Q2.⁻.

Radical formation from reaction of Q2 with ascorbate or GSH was studied by EPR using DMPO as a spin trap. The signal seen with Q2 and GSH (FIG. 8(B)) was predominantly the DMPO/.OH adduct, particularly strong after incubating at 37° C. for 15 min, but this is not evidence for production of a major component from free .OH radicals, since ethanol (10% v/v) was present. This would have scavenged .OH preferentially over DMPO, but only ˜15% of a signal (FIG. 8, species 2) identifiable with the DMPO/.CH(OH)CH₃ adduct was detected. The major component is instead thought to arise from decomposition of the DMPO/.OOH (superoxide) adduct (Finkelstein, E., et al. (1982) Mol. Pharmacol. 21, 262-265). The minor component (FIG. 8, species 3) is assigned to an uncharacterized oxidation product of the spin trap that is sometimes seen in other experiments involving strong oxidants (e.g. Reszka, K. J., and Chignell, C. F. (1995) Chem. Biol. Interact. 96, 223-234). No DMPO-trapped radicals were observed in solutions containing Q2, ethanol, and ascorbate, although ascorbate has been shown to reduce ethanol radical-DMPO adducts to spin-silent products (Stoyanovsky, D. A., et al. (1998), Free Radic. Biol. Med. 24, 132-138). Instead, the characteristic signal of the ascorbate radical was enhanced by Q2.

There are two key features of the oxygen consumption experiments involving Q2 or CA1 and GSH or ascorbate (FIGS. 5 and 6). First, more oxygen is depleted than CA1 or Q2 added, showing that turnover of oxygen is a chain reaction. Second, oxygen consumption requires Q2, either added initially (FIG. 5) or allowed to form via Q1 on standing after reaction of CA1 with HRP/H₂O₂ (FIG. 6). Numerous studies have been made of redox cycling of oxygen catalysed by quinones and GSH or ascorbate, or hydroquinones, demonstrating complex reaction pathways involving both quinone and ascorbate free radical intermediates (Brunmark, A., and Cadenas, E. (1989) Free Radic. Biol. Med. 7, 435-477; Goin, J., et al., (1991) Arch. Biochem. Biophys. 288, 386-396; O'Brien, P. J. (1991), Chem. Biol. Interact. 80, 1-41; Roginsky, V. A., et al., (1998) Free Radic. Res. 29, 115-125; Roginsky, V. A., et al. (1999) Chem. Biol. Interact. 121, 177-197; Roginsky, V., and Barsukova, T. (2000) J. Chem. Soc., Perkin Trans. 2, 1575-1582).

A key equilibrium is electron transfer between semiquinone(s) and oxygen:

Q.⁻+O₂→Q+O₂.⁻ (2)

which, by comparison with other ortho quinones, was expected to be over to the left for Q1 and Q2 (K₂<1) unless [O₂]>>[Q], since the mid-point electrode potentials Eₘ at pH ˜7.4 of simple ortho quinones are such that Eₘ(Q/Q.⁻)>Eₘ(O_{2[}1 M]/O₂.⁻) (45). (The semiquinones are largely dissociated at pH 7.4 since the pKₐs of the conjugate acids of methoxy-substituted ortho semiquinone radicals are ˜5.0 (Steenken et al., ibid) The value of Eₘ(Q2/Q2.⁻)=0.16 V vs. NHE suggested from the cyclic voltammetry experiments (FIG. 11(C)), the direct observation of rapid reaction of O₂.⁻ over tens of microseconds with only 30 μM Q2 and 1.25 mM O₂ (FIG. 10(B)), and the lack of reactivity of Q1.⁻ with oxygen over milliseconds (FIG. 9(B)) are all consistent with expectation that equilibrium (2) is indeed well over to the left with both Q1 and Q2. Studies with other ortho semiquinones have reached similar conclusions (Cooksey, C. J., et al., (1987) Free Radical Res. Commun. 4, 131-138; Kalyanaraman, B., et al. (1988) Arch. Biochem. Biophys. 266, 277-284). Loss of CA1 in suspensions of HL60 cells was observed provided SOD was added. This can be explained by SOD-catalysed removal of O₂.⁻ driving equilibrium (2) to the right, the semiquinone(s) being formed extracellularly from quinone(s) formed on intracellular oxidation of CA1 diffusing into the medium and generating semiquinone(s) via equilibrium (1).

Ascorbate reacts about as rapidly with O₂.⁻ as uncatalysed dismutation of the latter radical (Bielski, B. H. J., et al., (1985) J. Phys. Chem. Ref. Data 14, 1041-1100), so that ascorbate enhances oxygen turnover in the presence of Q2, as observed (FIG. 5). The EPR signal of the ascorbate radical was enhanced on adding Q2, reflecting the equilibrium:

Q+AscH⁻→Q.⁻+Asc.⁻(+H⁺) (3)

which is an additional route to semiquinone radicals. A further complexity is reduction of semiquinone(s) to hydroquinone(s) by ascorbate:

Q.⁻+AscH⁻(+H⁺)→QH₂+Asc.⁻ (4)

leading to a complex array of multiple equilibria, as discussed for simpler quinones by Roginsky et al., ibid. Because of the instability of Q1 it is not possible to characterize fully corresponding pathways in the present study, although the results clearly point to the possibility of enhanced oxidative stress following oxidation of CA1 in vivo. It is conceivable that this could involve in part hydroxyl radical formation, via reduction of H₂O₂ by semiquinones (Sushkov, D. G., et al., (1987) FEBS Lett. 225, 139-144; Kalyanaraman, B. et al., (1991) Arch. Biochem. Biophys. 286, 164-170; Li, B., et al. (1999) Chem. Res. Toxicol. 12, 1042-1049):

Q.⁻+H₂O₂→Q+.OH+OH⁻ (5)

as some evidence for reaction of .OH with ethanol was observed in solutions containing Q2 and GSH (FIG. 8).

Overall, the present study is consistent with the pathways summarized in FIG. 1. Oxidation of CA1 proceeds via a semiquinone radical to an ortho quinone Q1, highly reactive towards ascorbate and superoxide, reforming the hydroquinone, CA1. Q1 is reactive towards thiols, thus raising the possibility, not investigated in this work, of binding to protein thiols in competition with reaction with GSH. These reactions are themselves in competition with transformation of Q1 to Q2. The latter quinone catalyses oxygen consumption and thus has the potential to enhance cellular oxidative stress. In contrast, combretastatin A-4, although shown to be oxidized by enzyme-catalysed systems, does not stimulate oxygen turnover. The products of CA4 oxidation are likely to be dimers resulting from intermediate phenoxy radicals, similar to those arising from tyrosine oxidation (Jin, F., et al. (1993) J. Chem. Soc., Perkin Trans. 2 1583-1588).

In conclusion, the additional phenolic moiety in combretastatin A-1 compared to A-4 markedly changes the redox properties of the molecules and introduces completely different chemical functionality. The ortho quinones formed on oxidation of CA1 are key intermediates which may be synthesized and administered as therapeutic agents. In particular, identification of adducts of an unrelated ortho quinone not only with GSH but also with nucleotides (Qiu, S.-X., et al. (2004) Chem. Res. Toxicol. 17, 1038-1046) suggests that reactivity of CA1 metabolites with both proteins and nucleic acids offers an additional therapeutic mechanism. Both alkylation and oxidative stress have been correlated with the diverse roles of quinones in toxicology, with several examples including ortho quinone moieties (Bolton, J. L., et al. (2000) Chem. Res. Toxicol. 13, 135-160).

### EQUIVALENTS

Those skilled in the art will recognize, or be able to ascertain using no more than routine experimentation, numerous equivalents to the specific procedures described herein. Such equivalents are considered to be within the scope of the invention and are covered by the following claims. Various substitutions, alterations, and modifications may be made to the invention without departing from the spirit and scope of the invention as defined by the claims. Other aspects, advantages, and modifications are within the scope of the invention. The contents of all references, issued patents, and published patent applications cited throughout this application are hereby incorporated by reference. The appropriate components, processes, and methods of those patents, applications and other documents may be selected for the invention and embodiments thereof.

1. An isolated compound comprising the structure of Formula I: 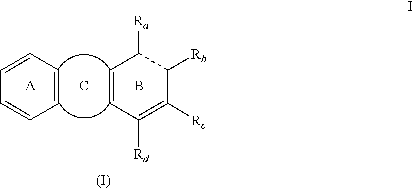
wherein:
(i) Ring A is independently substituted with one to four substituents selected from:
a C₁, C₂, C₃, C₄ or C₅ branched or straight-chain lower alkoxy, cycloalkoxy, heterocycloalkoxy, aryloxy, or lower alkanoyloxy group; or
halogen or trihaloalkyl; or
a C₁, C₂, C₃, C₄ or C₅ branched or straight chain lower alkyl, allyl, allyloxy, vinyl, or vinyloxy group; or
OH, or a C₁, C₂, C₃, C₄ or C₅ primary, secondary, or tertiary alcohol;
an NH₂ or an amino, lower alkylamino, arylamino, aralkylamino, cycloalkylamino, heterocycloamino, aroylamino, aralkanoylamino, amido, lower alkylamido, arylamido, aralkylamido, cycloalkylamido, heterocycloamido, aroylamido, or aralkanoylamido; or
a lower alkanoyl, thiol, sulfonyl, sulfonamide, nitro, nitrosyl, cyano, carboxy, aryl, or heterocyclo;
(ii) the dashed line of ring B is a single or double bond;
when the dashed line is a double bond, Rₐ and R_{b} are each independently:
a C₁, C₂, C₃, C₄ or C₅ branched or straight-chain lower alkoxy, cycloalkoxy, heterocycloalkoxy, aryloxy, or lower alkanoyloxy group; or
halogen or trihaloalkyl; or
a C₁, C₂, C₃, C₄ or C₅ branched or straight chain lower alkyl, allyl, allyloxy, vinyl, or vinyloxy group; or
OH or a C₁, C₂, C₃, C₄ or C₅ primary, secondary, or tertiary alcohol; or
NH₂ or an amino, lower alkylamino, arylamino, aralkylamino, cycloalkylamino, heterocycloamino, aroylamino, aralkanoylamino, amido, lower alkylamido, arylamido, aralkylamido, cycloalkylamido, heterocycloamido, aroylamido, or aralkanoylamido; or
lower alkanoyl, thiol, sulfonyl, sulfonamide, nitro, nitrosyl, cyano, carboxy, aryl, or heterocyclo;
with the proviso that when Rₐ is H, R_{b} is not OH;
when the dashed line is a single bond, Rₐ and R_{b} are each, independently, C═O; and R_{c} and R_{d} of Ring B are each, independently:
hydrogen, or a C₁, C₂, C₃, C₄ or C₅ branched or straight-chain lower alkoxy, cycloalkoxy, heterocycloalkoxy, aryloxy, or lower alkanoyloxy group; or
halogen or trihaloalkyl;
a C₁, C₂, C₃, C₄ or C₅ branched or straight chain lower alkyl, allyl, allyloxy, vinyl, or vinyloxy group; or
OH or C₁, C₂, C₃, C₄ or C₅ primary, secondary, or tertiary alcohol; or
NH₂ or an amino, lower alkylamino, arylamino, aralkylamino, cycloalkylamino, heterocycloamino, aroylamino, aralkanoylamino, amido, lower alkylamido, arylamido, aralkylamido, cycloalkylamido, heterocycloamido, aroylamido, or aralkanoylamido; or lower alkanoyl, thiol, sulfonyl, sulfonamide, nitro, nitrosyl, cyano, carboxy, aryl, or heterocyclo;
(ii) Ring C is an aromatic or non-aromatic, carbocyclic or heterocyclic, 5, 6, or 7 membered ring, optionally substituted with substituents selected from:
a C₁, C₂, C₃, C₄ or C₅ branched or straight-chain lower alkoxy, hydrogen, cycloalkoxy, heterocycloalkoxy, aryloxy, or lower alkanoyloxy group; or
halogen or trihaloalkyl; or
a C₁, C₂, C₃, C₄ or C₅ branched or straight chain lower alkyl, allyl, allyloxy, vinyl, or vinyloxy group; or
OH, or a C₁, C₂, C₃, C₄ or C₅ primary, secondary, or tertiary alcohol; or
NH₂ or an amino, lower alkylamino, arylamino, aralkylamino, cycloalkylamino, heterocycloamino, aroylamino, aralkanoylamino, amido, lower alkylamido, arylamido, aralkylamido, cycloalkylamido, heterocycloamido, aroylamido, or aralkanoylamido; or
lower alkanoyl, thiol, sulfonyl, sulfonamide, nitro, nitrosyl, cyano, carboxy, aryl, or heterocyclo.

2. An isolated compound comprising the structure of Formula I-A: 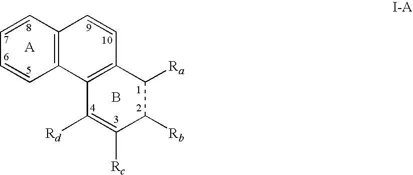
wherein:
(i) Ring A is independently substituted with one to four substituents selected from:
a C₁, C₂, C₃, C₄ or C₅ branched or straight-chain lower alkoxy, cycloalkoxy, heterocycloalkoxy, aryloxy, or lower alkanoyloxy group; or
halogen or trihaloalkyl; or
a C₁, C₂, C₃, C₄ or C₅ branched or straight chain lower alkyl, allyl, allyloxy, vinyl, or vinyloxy group; or
OH, or a C₁, C₂, C₃, C₄ or C₅ primary, secondary, or tertiary alcohol;
an NH₂ or an amino, lower alkylamino, arylamino, aralkylamino, cycloalkylamino, heterocycloamino, aroylamino, aralkanoylamino, amido, lower alkylamido, arylamido, aralkylamido, cycloalkylamido, heterocycloamido, aroylamido, or aralkanoylamido; or
a lower alkanoyl, thiol, sulfonyl, sulfonamide, nitro, nitrosyl, cyano, carboxy, aryl, or heterocyclo;
(ii) the dashed line of ring B is a single or double bond;
when the dashed line is a double bond, Rₐ and R_{b} are each independently:
a C₁, C₂, C₃, C₄ or C₅ branched or straight-chain lower alkoxy, cycloalkoxy, heterocycloalkoxy, aryloxy, or lower alkanoyloxy group; or
halogen or trihaloalkyl; or
a C₁, C₂, C₃, C₄ or C₅ branched or straight chain lower alkyl, allyl, allyloxy, vinyl, or vinyloxy group; or
OH or a C₁, C₂, C₃, C₄ or C₅ primary, secondary, or tertiary alcohol; or
NH₂ or an amino, lower alkylamino, arylamino, aralkylamino, cycloalkylamino, heterocycloamino, aroylamino, aralkanoylamino, amido, lower alkylamido, arylamido, aralkylamido, cycloalkylamido, heterocycloamido, aroylamido, or aralkanoylamido; or lower alkanoyl, thiol, sulfonyl, sulfonamide, nitro, nitrosyl, cyano, carboxy, aryl, or heterocyclo;
with the proviso that when Rₐ is H, R_{b} is not OH;
when the dashed line is a single bond, Rₐ and R_{b} are each, independently, C═O; and
R_{c} and R_{d} of Ring B are each, independently:
hydrogen, or a C₁, C₂, C₃, C₄ or C₅ branched or straight-chain lower alkoxy, cycloalkoxy, heterocycloalkoxy, aryloxy, or lower alkanoyloxy group; or
halogen or trihaloalkyl;
a C₁, C₂, C₃, C₄ or C₅ branched or straight chain lower alkyl, allyl, allyloxy, vinyl, or vinyloxy group; or
OH or C₁, C₂, C₃, C₄ or C₅ primary, secondary, or tertiary alcohol; or
NH₂ or an amino, lower alkylamino, arylamino, aralkylamino, cycloalkylamino, heterocycloamino, aroylamino, aralkanoylamino, amido, lower alkylamido, arylamido, aralkylamido, cycloalkylamido, heterocycloamido, aroylamido, or aralkanoylamido; or
lower alkanoyl, thiol, sulfonyl, sulfonamide, nitro, nitrosyl, cyano, carboxy, aryl, or heterocyclo; and
Ring C is independently substituted with one to two substituents selected from:
a C₁, C₂, C₃, C₄ or C₅ branched or straight-chain lower alkoxy, hydrogen, cycloalkoxy, heterocycloalkoxy, aryloxy, or lower alkanoyloxy group; or
halogen or trihaloalkyl; or
a C₁, C₂, C₃, C₄ or C₅ branched or straight chain lower alkyl, allyl, allyloxy, vinyl, or vinyloxy group; or
OH, or a C₁, C₂, C₃, C₄ or C₅ primary, secondary, or tertiary alcohol;
an NH₂ or an amino, lower alkylamino, arylamino, aralkylamino, cycloalkylamino, heterocycloamino, aroylamino, aralkanoylamino, amido, lower alkylamido, arylamido, aralkylamido, cycloalkylamido, heterocycloamido, aroylamido, or aralkanoylamido; or
a lower alkanoyl, thiol, sulfonyl, sulfonamide, nitro, nitrosyl, cyano, carboxy, aryl, or heterocyclo.

3. The compound of claim 2, wherein Ring A is substituted with one, two, three or four methoxy groups.

4. The compound of claim 2, wherein R_{c} and R_{d} are each, independently, hydrogen or a methoxy group.

5. The compound of claim 2, wherein the dashed line of ring B is a single bond;
Rₐ and R_{b} are both ═O;
Ring A is optionally substituted with one to five substituents selected from:
a C₁, C₂, C₃, C₄ or C₅ branched or straight-chain lower alkoxy, cycloalkoxy, heterocycloalkoxy, aryloxy, or lower alkanoyloxy group; or
a C₁, C₂, C₃, C₄ or C₅ branched or straight chain lower alkyl, allyl, allyloxy, vinyl, or vinyloxy group;
R_{c} is selected from:
a C₁, C₂, C₃, C₄ or C₅ branched or straight-chain lower alkoxy, cycloalkoxy, heterocycloalkoxy, aryloxy, or lower alkanoyloxy group; or
a C₁, C₂, C₃, C₄ or C₅ branched or straight chain lower alkyl, allyl, allyloxy, vinyl, or vinyloxy group; and
R_{d} is hydrogen.

6. The compound of claim 2, wherein the dashed line of ring B is a double bond;
Ring A is optionally substituted with one to five substituents selected from:
a C₁, C₂, C₃, C₄ or C₅ branched or straight-chain lower alkoxy, cycloalkoxy, heterocycloalkoxy, aryloxy, or lower alkanoyloxy group; or
a C₁, C₂, C₃, C₄ or C₅ branched or straight chain lower alkyl, allyl, allyloxy, vinyl, or vinyloxy group;
Rₐ and R_{b} are both OH;
R_{c} is selected from:
a C₁, C₂, C₃, C₄ or C₅ branched or straight-chain lower alkoxy, cycloalkoxy, heterocycloalkoxy, aryloxy, or lower alkanoyloxy group; or
a C₁, C₂, C₃, C₄ or C₅ branched or straight chain lower alkyl, allyl, allyloxy, vinyl, or vinyloxy group; and
R_{d} is hydrogen.

7. The compound of claim 2, wherein the compound of formula I-A is substituted with methoxy groups in the 3, 5, 6, and 7 positions.

8. An isolated compound comprising the structure of Formula I-B: 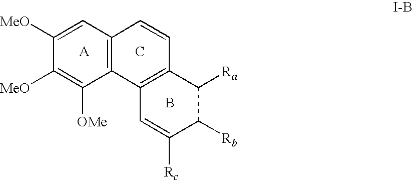
wherein:
the dashed line of ring B is a single or double bond;
when the dashed line is a double bond, Rₐ and R_{b} are each independently:
a C₁, C₂, C₃, C₄ or C₅ branched or straight-chain lower alkoxy, cycloalkoxy, heterocycloalkoxy, aryloxy, or lower alkanoyloxy group; or
a C₁, C₂, C₃, C₄ or C₅ branched or straight chain lower alkyl, allyl, allyloxy, vinyl, or vinyloxy group; or
OH or a C₁, C₂, C₃, C₄ or C₅ primary, secondary, or tertiary alcohol;
with the proviso that when Rₐ is H, R_{b} is not OH;
when the dashed line is a single bond, Rₐ and R_{b} are each, independently, C═O; and
R^{c} of Ring B is:
hydrogen, or a C₁, C₂, C₃, C₄ or C₅ branched or straight-chain lower alkoxy, cycloalkoxy, heterocycloalkoxy, aryloxy, or lower alkanoyloxy group; or
a C₁, C₂, C₃, C₄ or C₅ branched or straight chain lower alkyl, allyl, allyloxy, vinyl, or vinyloxy group; or
OH or C₁, C₂, C₃, C₄ or C₅ primary, secondary, or tertiary alcohol;
and
Ring C is independently substituted with one to two substituents selected from:
a C₁, C₂, C₃, C₄ or C₅ branched or straight-chain lower alkoxy, hydrogen, cycloalkoxy, heterocycloalkoxy, aryloxy, or lower alkanoyloxy group; or
halogen or trihaloalkyl; or
a C₁, C₂, C₃, C₄ or C₅ branched or straight chain lower alkyl, allyl, allyloxy, vinyl, or vinyloxy group; or
OH, or a C₁, C₂, C₃, C₄ or C₅ primary, secondary, or tertiary alcohol;
an NH₂ or an amino, lower alkylamino, arylamino, aralkylamino, cycloalkylamino, heterocycloamino, aroylamino, aralkanoylamino, amido, lower alkylamido, arylamido, aralkylamido, cycloalkylamido, heterocycloamido, aroylamido, or aralkanoylamido; or
a lower alkanoyl, thiol, sulfonyl, sulfonamide, nitro, nitrosyl, cyano, carboxy, aryl, or heterocyclo.

9. The compound of claim 8, wherein the compound of formula I-B is selected from the group consisting of 3,5,6,7-tetramethoxyphenanthrene-1,2-dione (1) and 3,5,6,7-tetramethoxyphenanthrene-1,2-diol (2).

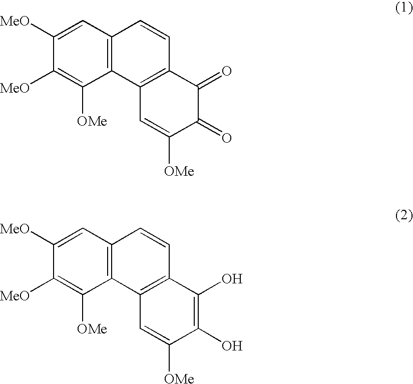

10. A method for selectively reducing blood flow to a tumor region and forming a ROS in a patient suffering from cancer, comprising administering a compound of any one of the preceding claims to said patient.

11. A method of inhibiting the proliferation of tumor cells in a patient suffering from cancer, comprising administering to the patient an effective amount of a compound of any of one claims 1-9.

12. A method of reducing blood flow in a patient suffering from a vascular proliferative disorder, comprising administering to the patient an effective amount of a compound of any one of claims 1-9.

13. A pharmaceutical composition comprising the compound of any one of claims 1-9 in a pharmaceutically acceptable carrier.

14. A kit comprising;
(a) a pharmaceutical composition comprising tablets, each comprising a compound of any one of claims 1-9 and a pharmaceutically acceptable carrier,
(b) a packaging material enclosing said pharmaceutical composition, and
(c) instructions for use of said pharmaceutical composition in the treatment of a subject in need thereof.
